SEQUENCE LISTING (18 sequences)

SEQ ID NO: 1 - protein, 985 aa, Homo sapiens
MQDIAFLSGGRGKDNAWIITFPENCNFRCIPEEVIAKVLTYLTSIARQNGSDSRFTIILDRRLDTWSSLKISLQKISASFPGNLHLVLVLRPTSFLQRTFTDIGFWFSQEDFMLKLPVVMLSSVSDLLTYIDDKQLTPELGGTLQYCHSEWIIFRNAIENFALTVKEMAQMLQSFGTELAETELPDDIPSIEEILAIRAERYHLLKNDITAVTKEGKILLTNLEVPDTEGAVSSRLECHRQISGDWQTINKLLTQVHDMETAFDGFWEKHQLKMEQYLQLWKFEQDFQQLVTEVEFLLNQQAELADVTGTIAQVKQKIKKLENLDENSQELLSKAQFVILHGHKLAANHHYALDLICQRCNELRYLSDILVNEIKAKRIQLSRTFKMHKLLQQARQCCDEGECLLANQEIDKFQSKEDAQKALQDIENFLEMALPFINYEPETLQYEFDVILSPELKVQMKTIQLKLENIRSIFENQQAGFRNLADKHVRPIQFVVPTPENLVTSGTPFFSSKQGKKTWRQNQSNLKIEVVPDCQEKRSSGPSSSLDNGNSLDVLKNHVLNELIQTERVYVRELYTVLLGYRAEMDNPEMFDLMPPLLRNKKDILFGNMAEIYEFHNDIFLSSLENCAHAPERVGPCFLERKDDFQMYAKYCQNKPRSETIWRKYSECAFFQECQRKLKHRLRLDSYLLKPVQRITKYQLLLKELLKYSKDCEGSALLKKALDAMLDLLKSVNDSMHQIAINGYIGNLNELGKMIMQGGFSVWIGHKKGATKMKDLARFKPMQRHLFLYEKAIVFCKRRVESGEGSDRYPSYSFKHCWKMDEVGITEYVKGDNRKFEIWYGEKEEVYIVQASNVDVKMTWLKEIRNILLKQQELLTVKKRKQQDQLTERDKFQISLQQNDEKQQGAFISTEETELEHTSTVVEVCEAIASVQAEANTVWTEASQSAEISEEPAEWSSNYFYPTYDENEEENRPLMRPVSEMALLY

SEQ ID NO: 2 - protein, 985 aa, Artificial Sequence; identical to SEQ ID NO: 1
  Protein sequence of human Dbl3Y570D

SEQ ID NO: 3 - DNA, 2961 bp, Homo sapiens
cttaatgcaagacatcgccttcttgtctggtggccggggaaaggacaatgcttggatcattacgtttccagaaaactgtaattttagatgtataccagaggaagtaatagcaaaagtacttacttacctgacatctattgcaaggcaaaatggatcagactcccggtttaccattattctggatcgaagattggatacatggtcttctctcaaaatctctctccaaaaaatctcggcttccttccctgggaacttgcacttggttttggttttacgtcctaccagctttcttcaacgaacgttcacagacattggattttggtttagtcaggaggattttatgcttaaattaccagttgttatgctgagctcagttagtgatttgctgacatacattgatgacaagcaattaacccctgagttaggcggcaccttgcagtactgccacagtgaatggatcatcttcagaaatgctatagaaaattttgccctcacagtgaaagaaatggctcagatgttacagtcctttggaactgaactggctgagacagaactaccagatgatattccctcaatagaagaaattctggcaattcgtgctgaaaggtatcatctgttgaagaatgatattacagctgtaaccaaagaaggaaaaattctgctaacaaatctggaagtgcctgacactgaaggagctgtcagttcaagactagaatgtcatcggcaaataagtggtgactggcaaactattaataagttgctgactcaagtacatgatatggaaacagcttttgatggattttgggaaaaacatcaattaaaaatggagcagtatctgcaactatggaagtttgagcaggattttcaacagcttgtgactgaagttgaatttctattaaaccaacaagcagaactggctgatgtaacagggactatagctcaagtaaaacaaaaaataaaaaaattggaaaacttagatgaaaattctcaggagctattatcaaaggcccagtttgtgatattacatggacacaagcttgcagcaaatcaccattatgcacttgatttaatctgccagaggtgcaatgagctacgttacctttctgatattttggttaatgagataaaagcaaaacggatacaactcagcaggaccttcaaaatgcataaactcctacagcaggctcgtcaatgctgtgatgaaggggaatgtcttctagctaatcaggaaatagataagtttcagtctaaagaagatgctcagaaagctctccaagacattgaaaattttcttgaaatggctctaccctttataaattatgaacctgaaacactgcagtatgaatttgatgtaatattatctcctgagcttaaggttcaaatgaagactatacaactcaagcttgaaaacattcgaagtatatttgagaaccagcaggctggtttcaggaacctggcagataagcatgtgaggccaatccaatttgtggtacccacacctgaaaatttggtcacatctgggacaccatttttttcatctaaacaagggaagaagacttggagacaaaatcagagcaacttaaaaattgaagtggtgcctgattgtcaggagaagagaagttctggtccatcctccagtttggacaatggcaatagcttggatgttttaaagaaccacgtactaaatgaactgatacagactgagagagtttatgttcgagaactgtatactgttttgttgggttatagagcggagatggataatccagagatgtttgatcttatgccacctctcctgagaaataaaaaggacattctctttggaaacatggcagaaatatatgaattccataacgacattttcttgagcagcctggaaaattgtgctcatgctccagaaagagtgggaccttgtttcctggaaaggaaggatgattttcagatgtatgcaaaatattgtcagaataagcccagatcagaaacaatttggaggaagtattcagaatgcgcatttttccaggaatgtcaaagaaagttaaaacacagacttagactggattcctatttactcaaaccagtgcaacgaatcactaaatatcagttattgttgaaggagctattaaaatatagcaaagactgtgaaggtctgctctgttgaagaaggcactcgatgcaatgctggatttactgaagtcagttaatgattctatgcatcagattgcaataaatggctatattggaaacttaaatgaactgggcaagatgataatgcaaggtggattcagcgtttggatagggcacaagaaaggtgctacaaaaatgaaggatttggctagattcaaaccaatgcagcgacaccttttcttgtatgaaaaagccattgttttttgcaaaaggcgtgttgaaagtggagaaggctctgacagatacccgtcatacagttttaaacactgttggaaaatggatgaagttggaatcactgaatatgtaaaaggtgataaccgcaagtttgaaatctggtatggtgaaaaggaagaagtttatattgtccaggcttctaatgtagatgtgaagatgacgtggctaaaagaaataagaaatattttgttgaagcagcaggaacttttgacagttaaaaaaagaaagcaacaggatcaattaacagaacgggataagtttcagatttctcttcagcagaatgatgaaaagcaacagggagcttttataagtactgaggaaactgaattggaacacaccagcactgtggtggaggtctgtgaggcaattgcgtcagttcaggcagaagcaaatacagtttggactgaggcatcacaatctgcagaaatctctgaagaacctgcggaatggtcaagcaactatttctaccctacttatgatgaaaatgaagaagaaaataggcccctcatgagacctgtgtcggagatggctctcctatattga

SEQ ID NO: 4 - DNA, 2961 bp, Artificial Sequence
  DNA sequence encoding human Dbl3Y570D
cttaatgcaagacatcgccttcttgtctggtggccggggaaaggacaatgcttggatcattacgtttccagaaaactgtaattttagatgtataccagaggaagtaatagcaaaagtacttacttacctgacatctattgcaaggcaaaatggatcagactcccggtttaccattattctggatcgaagattggatacatggtcttctctcaaaatctctctccaaaaaatctcggcttccttccctgggaacttgcacttggttttggttttacgtcctaccagctttcttcaacgaacgttcacagacattggattttggtttagtcaggaggattttatgcttaaattaccagttgttatgctgagctcagttagtgatttgctgacatacattgatgacaagcaattaacccctgagttaggcggcaccttgcagtactgccacagtgaatggatcatcttcagaaatgctatagaaaattttgccctcacagtgaaagaaatggctcagatgttacagtcctttggaactgaactggctgagacagaactaccagatgatattccctcaatagaagaaattctggcaattcgtgctgaaaggtatcatctgttgaagaatgatattacagctgtaaccaaagaaggaaaaattctgctaacaaatctggaagtgcctgacactgaaggagctgtcagttcaagactagaatgtcatcggcaaataagtggtgactggcaaactattaataagttgctgactcaagtacatgatatggaaacagcttttgatggattttgggaaaaacatcaattaaaaatggagcagtatctgcaactatggaagtttgagcaggattttcaacagcttgtgactgaagttgaatttctattaaaccaacaagcagaactggctgatgtaacagggactatagctcaagtaaaacaaaaaataaaaaaattggaaaacttagatgaaaattctcaggagctattatcaaaggcccagtttgtgatattacatggacacaagcttgcagcaaatcaccattatgcacttgatttaatctgccagaggtgcaatgagctacgttacctttctgatattttggttaatgagataaaagcaaaacggatacaactcagcaggaccttcaaaatgcataaactcctacagcaggctcgtcaatgctgtgatgaaggggaatgtcttctagctaatcaggaaatagataagtttcagtctaaagaagatgctcagaaagctctccaagacattgaaaattttcttgaaatggctctaccctttataaattatgaacctgaaacactgcagtatgaatttgatgtaatattatctcctgagcttaaggttcaaatgaagactatacaactcaagcttgaaaacattcgaagtatatttgagaaccagcaggctggtttcaggaacctggcagataagcatgtgaggccaatccaatttgtggtacccacacctgaaaatttggtcacatctgggacaccatttttttcatctaaacaagggaagaagacttggagacaaaatcagagcaacttaaaaattgaagtggtgcctgattgtcaggagaagagaagttctggtccatcctccagtttggacaatggcaatagcttggatgttttaaagaaccacgtactaaatgaactgatacagactgagagagttgatgttcgagaactgtatactgttttgttgggttatagagcggagatggataatccagagatgtttgatcttatgccacctctcctgagaaataaaaaggacattctctttggaaacatggcagaaatatatgaattccataacgacattttcttgagcagcctggaaaattgtgctcatgctccagaaagagtgggaccttgtttcctggaaaggaaggatgattttcagatgtatgcaaaatattgtcagaataagcccagatcagaaacaatttggaggaagtattcagaatgcgcatttttccaggaatgtcaaagaaagttaaaacacagacttagactggattcctatttactcaaaccagtgcaacgaatcactaaatatcagttattgttgaaggagctattaaaatatagcaaagactgtgaaggtctgctctgttgaagaaggcactcgatgcaatgctggatttactgaagtcagttaatgattctatgcatcagattgcaataaatggctatattggaaacttaaatgaactgggcaagatgataatgcaaggtggattcagcgtttggatagggcacaagaaaggtgctacaaaaatgaaggatttggctagattcaaaccaatgcagcgacaccttttcttgtatgaaaaagccattgttttttgcaaaaggcgtgttgaaagtggagaaggctctgacagatacccgtcatacagttttaaacactgttggaaaatggatgaagttggaatcactgaatatgtaaaaggtgataaccgcaagtttgaaatctggtatggtgaaaaggaagaagtttatattgtccaggcttctaatgtagatgtgaagatgacgtggctaaaagaaataagaaatattttgttgaagcagcaggaacttttgacagttaaaaaaagaaagcaacaggatcaattaacagaacgggataagtttcagatttctcttcagcagaatgatgaaaagcaacagggagcttttataagtactgaggaaactgaattggaacacaccagcactgtggtggaggtctgtgaggcaattgcgtcagttcaggcagaagcaaatacagtttggactgaggcatcacaatctgcagaaatctctgaagaacctgcggaatggtcaagcaactatttctaccctacttatgatgaaaatgaagaagaaaataggcccctcatgagacctgtgtcggagatggctctcctatattga

SEQ ID NO: 5 - DNA, 1614 bp, Homo sapiens
tattgtgcaaataagtgctcactccaaattagtggtatatttattgaagtttaatattgtgtttgtgatacagaagtatttgctttaattctaaataaaaattttatgcttttattgctggtttaagaagatttggattatccttgtactttgaggagaagtttcttatttgaaatattttggaaacaggtcttttaatgtggaaagatagatattaatctcctcttctattactctccaagatccaacaaaagtgattataccccccaaaatatgatggtagtatcttatactaccatcattttataggcatagggctcttagctgcaaataatggaactaactctaataaagcagaacgcaaatattgtaaatattagagagctaacaatctctgggatggctaaaggatggagcttggaggctacccagccagtaacaatattccgggctccactgttgaatggagacactacaactgccttggatgggcagagatattatggatgctaagccccaggtgctaccattaggacttctaccactgtccctaacgggtggagcccatcacatgcctatgccctcactgtaaggaaatgaagctactgttgtatatcttgggaagcacttggattaattgttatacagttttgttgaagaagacccctagggtaagtagccataactgcacactaaatttaaaattgttaatgagtttctcaaaaaaaatgttaaggttgttagctggtatagtatatatcttgcctgttttccaaggacttctttgggcagtaccttgtctgtgctggcaagcaactgagacttaatgaaagagtattggagatatgaatgaattgatgctgtatactctcagagtgccaaacatataccaatggacaagaaggtgaggcagagagcagacaggcattagtgacaagcaaagatatgcagaatttcattctcagcaaatcaaaagtcctcaacctggttggaagaatattggcactgaatggtatcaataaggttgctagagagggttagaggtgcacaatgtgcttccataacattttatacttctccaatcttagcactaatcaaacatggttgaatactttgtttactataactcttacagagttataagatctgtgaagacagggacagggacaatacccatctctgtctggttcataggtggtatgtaatagatatttttaaaaataagtgagttaatgaatgagggtgagaatgaaggcacagaggtattagggggaggtgggccccagagaatggtgccaaggtccagtggggtgactgggatcagctcaggcctgacgctggccactcccacctagctcctttctttctaatctgttctcattctccttgggaaggattgaggtctctggaaaacagccaaacaactgttatgggaacagcaagcccaaataaagccaagcatcagggggatctgagagctgaaagcaacttctgttccccctccctcagctgaaggggtggggaagggctcccaaagccataactccttttaagggatttagaaggcataaaaaggcccctggctgagaacttccttcttcattctgcagttgg

SEQ ID NO: 6 - DNA, 761 bp, Artificial Sequence
  Optimised RPE65 promoter fragment
agatcttcgaaatactctcagagtgccaaacatataccaatggacaagaaggtgaggcagagagcagacaggcattagtgacaagcaaagatatgcagaatttcattctcagcaaatcaaaagtcctcaacctggttggaagaatattggcactgaatggtatcaataaggttgctagagagggttagaggtgcacaatgtgcttccataacattttatacttctccaatcttagcactaatcaaacatggttgaatactttgtttactataactcttacagagttataagatctgtgaagacagggacagggacaatacccatctctgtctggttcataggtggtatgtaatagatatttttaaaaataagtgagttaatgaatgagggtgagaatgaaggcacagaggtattagggggaggtgggccccagagaatggtgccaaggtccagtggggtgactgggatcagctcaggcctgacgctggccactcccacctagctcctttctttctaatctgttctcattctccttgggaaggattgaggtctctggaaaacagccaaacaactgttatgggaacagcaagcccaaataaagccaagcatcagggggatctgagagctgaaagcaacttctgttccccctccctcagctgaaggggtggggaagggctcccaaagccataactccttttaagggatttagaaggcataaaaaggcccctggctgagaacttccttcttcattctgcagttgg

SEQ ID NO: 7 - DNA, 624 bp, Artificial Sequence
  Optimised BEST1 promoter fragment
gaattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagacc

SEQ ID NO: 8 - DNA, 22 bp, Artificial Sequence
  MRCKbeta sequence targeted by CRISPR Cas9
cctggacgggccgtggcgcaac

SEQ ID NO: 9 - DNA, 21 bp, Artificial Sequence
  MRCKbeta sequence targeted by CRISPR Cas9
acaccgagtgcagccactcgg

SEQ ID NO: 10 - DNA, 25 bp, Artificial Sequence
  Dbl3 sequence targeted by CRISPR Cas9
tggagatcgaagactggatacatgg

SEQ ID NO: 11 - RNA, 21 nt, Artificial Sequence
  Sequence in human N-WASP targeted by siRNA
cagauacgacaggguauccaa

SEQ ID NO: 12 - RNA, 21 nt, Artificial Sequence
  Sequence in human N-WASP targeted by siRNA
uagagagggugcucagcuaaa

SEQ ID NO: 13 - RNA, 19 nt, Artificial Sequence
  Sequence in porcine Cdc42 targeted by siRNA
gaugaccccucuacuauug

SEQ ID NO: 14 - RNA, 19 nt, Artificial Sequence
  Sequence in porcine Dbl3 targeted by siRNA
aagacaucgccuuccuguc

SEQ ID NO: 15 - RNA, 19 nt, Artificial Sequence
  Sequence in porcine Dbl3 targeted by siRNA
auaccuggucuucucucaa

SEQ ID NO: 16 - RNA, 19 nt, Artificial Sequence
  Sequence in porcine MRCKbeta targeted by siRNA
cgagaagacuucgaaauaa

SEQ ID NO: 17 - RNA, 19 nt, Artificial Sequence
  Sequence in porcine MRCKbeta targeted by siRNA
agagaagacuuugaaauau

SEQ ID NO: 18 - protein, 9 aa, Artificial Sequence
  Conserved Dbl activation motif
TERVYVREL

(19) United States
(12) Patent Application Publication
(10) Pub. No.: US 2023/0135501 A1
(43) Pub. Date: May 4, 2023

(54) GENE THERAPY

(71) Applicant: UCL BUSINESS LTD, London (GB)

(72) Inventors: Ceniz ZIHNI, London (GB); Maria Susana BALDA, London (GB); Karl MATTER, London (GB)

(21) Appl. No.: 17/798,796

(22) PCT Filed: Feb. 18, 2021
(86) PCT No.: PCT/GB2021/050402

§ 371 Date: Aug. 10, 2022

(30) Foreign Application Priority Data
Feb. 18, 2020 (GB) 2002202.6

(51) Int. Cl. C07K 14/47; C12N 15/86; A61K 48/00; A61P 27/02

(52) U.S. Cl. CPC C07K 14/4705; C12N 15/86; A61K 48/0058; A61P 27/02; C12N 2750/14143; C12N 2750/14145; C07K 2319/33

(57) ABSTRACT

The invention relates to the use of vectors to improve vision by restoring RPE phagocytosis of photoreceptor outer segments in a patient suffering from retinal dysfunction and/or degeneration.

### FIELD OF THE INVENTION

The invention relates to the use of vectors to improve vision by restoring RPE phagocytosis of photoreceptor outer segments in a patient suffering from retinal dysfunction and/or degeneration.

### BACKGROUND OF THE INVENTION

The retinal pigment epithelium (RPE) forms a monolayer of highly specialized neuroectodermally derived pigmented cells located between the neurosensory retina and the vascular choroid. RPE cells interdigitate with photoreceptor outer segments (POS) and provide essential support functions for the neural retina and, in particular, photoreceptors. These functions include participation in the visual cycle, phagocytosis of shed POS, maintenance of the outer blood-retinal barrier, secretion of neurotrophic, inflammatory, and vasculotrophic growth factors, water transport out of the subretinal space, and regulation of bidirectional ion and metabolic transport between the retina and choroid.

POS are rapidly renewed to ensure maximum photosensitivity. Older disks are displaced distally and eventually shed in packets from the tip of the POS, where they are phagocytosed by the RPE. Thus, photoreceptor cells maintain a roughly constant length by continuously generating new outer segments from their base. The prompt and efficient clearance of POS by receptor-mediated phagocytosis is essential for both long-term viability and functionality of photoreceptors.

Mechanistically, phagocytosis comprises the steps of attachment and engulfment of POS. Following phagocytosis, POS are degraded. Although some components involved in general phagocytosis are also involved in POS phagocytosis, RPE cells require a specialized set of machinery. With the development of modern molecular and biochemical techniques, including polarized RPE cell cultures, several important components of the POS phagocytosis pathway have been identified.

In live cells, components of the plasma membrane are asymmetrically distributed, with phospholipids containing terminal primary amino groups, such as phosphatidylserine (PS) and phosphatidylethanolamine (PE), localized to the cytoplasmic surface. During apoptosis, this asymmetry is disrupted, and patches of extracellularly exposed PS are thought to act as phagocytosis signals recognized by macrophages.

RPE cells recognize shed POS using a similar mechanism. However, external PS patches do not interact with receptors on the RPE directly but via soluble proteins; milk fat globule-EGF-factor 8 (MFG-E8), growth arrest-specific 6 (Gas6) and serum protein S. These soluble proteins then in turn bind to receptors on the RPE surface. MFG-E8 acts as a αVβ5 integrin ligand and is required for diurnal phagocytosis of POS. Gas6 and serum protein S also bind to PS on the POS surface, and are recognized by the tyrosine kinase MerTK, which regulates ingestion of the POS; mutations in this gene are responsible for defects in phagocytosis and the onset of retinitis pigmentosa in humans and retinal dystrophy in the RCS rat. CD36, a transmembrane glycoprotein, is described as functioning downstream and independently of αVβ5 integrin to control the rate of POS uptake. Focal adhesion kinase (FAK) is a non-receptor tyrosine kinase that resides at sites of integrin clustering known as focal adhesions. FAK also is essential for the engulfment of POS during RPE phagocytosis. POS binding to the αVβ5 receptor mobilizes a pool of FAK to the apical RPE and causes an autophosphorylation event. This activation of FAK ultimately results in phosphorylation of MerTK.

However, although the role of the RPE in POS recognition and engulfment is recognized, how the receptors and their signaling partners transmit the POS stimulus to power remodeling of the apical membrane, its underlying cortex, and POS internalization, is not known. In particular, little is known about the identity of the guanine nucleotide exchange factor (GEF) or GTPases required for actin polymerization or actomyosin contractility. Phagocytic activity regulates the mechanisms that control the metabolic activity of cells and gene expression; however, the components of the phagocytic machinery important for this regulation are not known. Despite its central importance to a broad range of biological processes, the precise mechanism of interplay between F-actin polymerization and actomyosin contractility that guides the cortex to power cell shape changes is also poorly understood. In particular, how actin polymerization and contractility are coupled and coordinated at a single location in a complex biological system, such as the polarized RPE of the retina, is not known.

The RPE is the source and the target of many retinal degenerative diseases. Defects in RPE function, such as phagocytosis, can affect the integrity and viability of neighbouring cells, and primarily photoreceptors. Depending on which part of the machinery is flawed, phenotypes can either appear early in life, such as retinitis pigmentosa or Usher syndrome, or develop with age, such as age-related macular degeneration, affecting first either the peripheral or the central retina.

Gene therapy is currently being tested to rescue phagocytosis of POS by re-expressing functional MerTK (Ghazi et al, 2016). However, the potential therapeutic effectiveness is limited to retinitis pigmentosa caused by mutations in MerTK. RPE phagocytosis is also deficient in age-related macular degeneration (AMD), a common disease that leads to reduced and loss of vision. There are two forms of the disease: dry and wet AMD, with the latter being associated with extensive neovascularization and vascular leakage. There are currently no effective treatments for dry AMD. The wet form of the disease can be treated with anti-VEGF therapeutics; however, long-term treatment often fails due to side-effects such as induction of subretinal fibrosis and RPE dysfunction (Maguire et al., 2016). Cell transplantation is also being trialled for wet AMD (da Cruz et al., 2018), however, whether it will work in the long term is not known yet, and it is an expensive approach.

There is a need for a more universal, effective therapy towards repairing a defective RPE, but the principal problem hindering such an approach is a lack of knowledge of the molecular mechanisms that control critical steps of activation of phagocytosis.

The invention described herein solves the above-mentioned problem.

### SUMMARY OF THE INVENTION

The present inventors have identified a conserved apical signalling plaque in the RPE that controls the mechanics of phagocytosis. In particular, the inventors have shown that the RPE forms novel apical signalling plaques (PASPs) in response to POS adhesion to its receptors by recruiting a MerTK tyrosine kinase activated Dbl3/Cdc42/N-WASP/MRCKβ signalling network.

PASPs serve as a cortical Rho GTPase signalling platform which couple and coordinate actin polymerisation and contractility at a single location within a complex biological system. The interdependence of actin polymerisation and contractility during cup reorganisation creates the ideal conditions for the spatial control of actin cytoskeleton dynamics and actomyosin contractility.

Assembly of PASPs is stimulated by POS adhesion to integrin αVβ5, which triggers the recruitment of the Dbl3-based signalling hub. Engagement and activation of MerTK then triggers phosphorylation and stimulation of the guanine nucleotide exchange factor (GEF) Dbl3, activation of Cdc42, and coupling of N-WASP- and MRCKβ/myosin-II-dependent membrane remodelling leading to the maturation of PASPs to phagocytic cups and the internalisation of POS.

Dbl3 is a Dbl/MCF2 isoform that regulates epithelial morphogenesis, positioning of cell junctions, and the apical-lateral border, as well as apical domain differentiation and size (Zihni et al., 2014). However, the involvement of Dbl3 and MRCK in phagocytosis generally, and specifically phagocytosis of POS, has not previously been described.

Although the role of Cdc42 at basolateral membrane cell-cell and cell-substrate adhesion sites has been studied, Cdc42 was not known to be involved in POS phagocytosis. Previous work on Rho GTPases in RPE phagocytosis failed to identify Cdc42 activation in response to POS binding; hence, a role for Cdc42 in phagocytosis was excluded (Mao et al., 2015). Furthermore, to date, no Cdc42 GEFs or Cdc42-specific effector molecules have been reported to be required for or to participate in POS phagocytosis. Subsequently it had been assumed that the Cdc42 pathway was absent during apical RPE remodelling during phagocytosis, accounting for the large void in information regarding the critical initial steps of RPE phagocytosis.

The inventors have shown that inactivation of the Dbl3 pathway induces proinflammatory signalling. Hence, deficiency in the Dbl3/MRCK mechanism not only leads to loss of phagocytosis but also inflammatory signalling. Inflammatory signalling is also a hallmark of AMD.

According to the invention, the apical signalling plaque can be engineered to restore RPE function. For example, expression of active Dbl3 mimicking a phosphorylated form has been shown to rescue phagocytosis in genetically phagocytosis-deficient RPE cells by rescuing PASP conversion to phagocytic cup. Such phagocytosis-deficient RPE cells may be derived from an individual with retinitis pigmentosa. Thus, Dbl3 is a promising therapeutic target to rescue diseased RPE and, thereby, attenuate retinal degeneration.

The present invention therefore provides:

an expression construct comprising a promoter operably linked to a nucleic acid sequence encoding a Dbl3 polypeptide.

Also provided is:

a vector comprising an expression construct of the invention, or a host cell comprising a vector of the invention;

a Dbl3 polypeptide, or a nucleic acid sequence encoding a Dbl3 polypeptide, or an expression construct of the invention, or a vector of the invention, for use in a method for treating retinal dysfunction and/or degeneration;

a vector comprising a nucleic acid sequence or an expression construct encoding a gene product that rescues phagocytosis of photoreceptor outer segments, for use in a method of treating retinal dysfunction and/or degeneration;

the use of a vector of the invention in the manufacture of a medicament for the treatment of an inherited retinal disorder or dystrophy, such as retinitis pigmentosa, or macular degeneration; and

a method of improving vision in a patient with retinal dysfunction and/or degeneration by introducing into the RPE a nucleic acid encoding a Dbl3 polypeptide.

### DESCRIPTION OF THE DRAWINGS

FIG. 1 shows that MRCKβ is required for RPE phagocytosis and retinal tissue homeostasis. Schematic illustrations of the RPE/photoreceptor architecture and subretinal delivery of lentiviral vectors to the RPE are shown in FIGS. 1a and 1b. Confocal immunofluorescence analysis of retinal sections from mice injected with control (FIG. 1c-left panel) or MRCKβ knockout (FIG. 1c—right panel) vectors reveal no significant difference in the overall retinal structure after 7 days but reduced apical/basal ratio F-actin staining in the MRCKβ deficient RPE (FIG. 1d). Transmission electron microscopic analysis shows that RPE from control CRISPR-vector-injected mice internalized POS whereas POS internalization in MRCKβ CRISPR-vector injected mice was inhibited, and POS fragments accumulated in the extracellular space (FIG. 1e to 1i). Transmission electron microscopic analysis of apical microvilli architecture in control CRISPR-vector-injected and MRCKβ CRISPR-vector injected mice are shown in FIGS. 1j to 1m. The quantifications show that microvilli from the RPE of control mice display ordered packing and morphology whereas knockout of MRCKβ leads to a disordered appearance of microvilli with irregular thickness. In this figure, quantifications show means±1SD, n=3, p-values derived from t-tests.

FIG. 2 shows that MRCKβ is required for POS-induced phagocytic membrane remodelling in vitro. siRNA-mediated knock-down of MRCKβ in primary porcine RPE cultures results in POS-induced apical membrane remodelling defects and inhibited phagocytosis as determined by confocal z-sections of porcine primary RPE cells stained with Atto647-Phalloidin (FIG. 2a-d). In this figure, POS were labelled with FITC; their positions relative to the cell (inside or outside) are pointed out by white arrows. Inhibition of MRCKβ kinase activity during the POS incubation with BDP5290 results in membrane remodelling and phagocytosis defects similar to siRNA-mediated knockdown (FIG. 2e-g). Transmission electron microscopy of primary porcine RPE cells using GOLD-labelled POS further reveals MRCK kinase inhibition using BDP5290 results in an inhibition of phagocytosis FIG. 2h-i). Internalized Gold-labelled POS in phagosomes in control samples are highlighted by black arrows. The quantification in FIGS. 2b, 2d, 2g are based on n=3 independent experiments and shown are the data points, means±1SD (top of bar), the total number of cells analysed for each type of sample across all experiments, and p-values derived from t-tests. siRNA-mediated knock-down of MRCKβ in human THP-1-derived macrophages showed that phagocytosis in response to opsonized zymosan-594 dependent Fc-receptor stimulation is inhibited (FIGS. 2j and 2k; white arrows represent the position of zymosan-594 particles). MRCKβ functions after cup morphogenesis in this system. The quantifications are based on n=3 independent experiments and shown are the data points, means±1SD (top of bar), the total number of cells analysed for each type of sample across all experiments, and p-values derived from t-tests. FIG. 2l shows a schematic model of the proposed role of MRCKβ in RPE and Fc-receptor-mediated macrophage phagocytosis. In the RPE, MRCKβ is required from the onset for cup assembly and encirclement of POS (i) and myosin contraction-dependent internalization of POS (ii). In macrophages, MRCKβ is not required for stage (i) but stage (ii), demonstrating conservation between topologically and mechanistically diverse systems.

FIG. 3 shows that MRCK and N-WASP form a dual effector mechanism to generate contractile rings/cups. FIG. 3a show a time course of POS-induced pseudopod assembly and remodelling which results in myosin motor activation. In porcine primary RPE cultures, following POS adhesion for 30 minutes, pseudopod forming cups enriched in pMLC form a contractile ring around the particle. FIG. 3b illustrates the cooperation between membrane remodelling and actomyosin contractility at cups that pull POS into the cell as quantified in FIGS. 3c and 3d. FIG. 3e-k demonstrate the requirement of MRCK in contractile cup centring of POS. MRCKβ inhibition prevented myosin activation and cup maturation. Inhibition of myosin-II motor activity using blebbistatin results in similar membrane remodelling and POS centring defects and was reversed following blebbistatin washout only when MRCK was active (FIGS. 3l to 3m). Inhibition of N-WASP activity inhibits pseudopod assembly or results in immature pseudopods with inhibited pMLC (FIGS. 3n to 3r). All quantifications are based on n=3 independent experiments. Shown are the data points, means±1SD, the total number of cells analysed for each type of sample across all experiments, and p-values derived from t-tests. FIG. 3s shows a schematic illustration of proposed model of MRCK and N-WASP interplay during de novo cup assembly. Note that N-WASP-dependent F-actin polymerization is required to provide the framework for pMLC activity and cup maturation.

FIG. 4 shows that POS-membrane adhesion generates Dbl3-activated Cdc42 plaques. Incubation of porcine primary RPE cells with POS-FITC for 30 minutes led to the formation of PASPs harbouring the Cdc42 GEF Dbl3, active Cdc42-GTP and the Cdc42 effectors MRCKβ and N-WASP (FIGS. 4a and 4b). Cdc42 signalling components co-localized with POS at PASPS and at contact sites in cups (FIG. 4c, 4d; depicted in white using Zeiss 700 imaging software based on the Pearson's Coefficient). Quantification of pMLC staining reveal progressively increasing myosin-II activation during cup maturation (FIG. 4e). FIG. 4f shows a schematic illustration of PASP to cup progression that relies on contractility. Contractility proceeds to pull POS into the cell body. Exogenous expression of Dbl3-myc in ARPE-19 cells strongly enhances POS-induced pseudopods and phagocytosis that is dependent on MRCKβ, N-WASP and Cdc42 (FIGS. 4g to 4m). Dbl3-myc stimulated pseudopods require N-WASP activity (FIG. 4l). FIG. 4m shows a schematic illustration of proposed model of Dbl3 orchestrated coordination of N-WASP and MRCKβ activity during contractile cup maturation. Dbl3 signalling is conserved in macrophage phagocytosis (FIG. 4n and FIG. 4o). FIG. 4p shows a schematic diagram illustrating the conserved Dbl3-MRCK signalling module in macrophage phagocytosis. All quantifications are based on n=3 independent experiments and show the data points, means±1SD, the total number of cells analysed for each type of sample across all experiments, and p-values derived from t-tests.

FIG. 5 shows that Dbl3 is activated by MerTK signalling. Integrin αVβ5 and MerTK transmembrane receptors co-localize with POS at PASPs in primary porcine RPE cells (FIG. 5a, 5b). Stimulating primary porcine RPE cells with POS+Gas-6 in serum-deprived conditions promotes pseudopod formation, which is N-WASP but not MRCK dependent (FIGS. 5c and 5e). Note that MRCK is required for pMLC activity at cups (FIG. 5d). siRNA-mediated knockdown of Dbl3 attenuates POS/Gas-6-dependent pseudopod pMLC and POS centring (FIG. 5f). Schematic model of proposed mechanism of Dbl3 activation via tyrosine kinase phosphorylation and regulation of integrin αVβ5-POS signalling (FIG. 5g). FIG. 5h-j shows that Dbl3-Cdc42-MRCK signalling affects actomyosin-dependent clustering of integrin αVβ5. POS-stimulation leads to tyrosine phosphorylation of Dbl3 (FIGS. 5l to 5o). The tyrosine phosphorylation motif in the DH domain of Dbl3 (FIG. 5l) is conserved in a diverse range of mammalian species (FIG. 5m). Exogenous expression of phosphorylation resistant Dbl3Y570F-myc or Dbl3-myc wild type in the presence of a c-Src inhibitor blocks phagocytosis in ARPE-19 cells, whereas Dbl3Y570D-myc stimulates POS internalization (FIGS. 5n and 5o). All quantifications are based on n=3 independent experiments and show the data points, means±1SD, the total number of cells analysed for each type of sample across all experiments, and p-values derived from t-tests.

FIG. 6 shows the rescue of phagocytosis by active Dbl3 in RP patient-derived RPE cells. FIG. 6a is a fundus image taken from the RP patient's right eye showing the retinal vessels (black arrows) and macula area (white circle). FIG. 6b shows a red free image of the macula, wherein the arrow represents the position of the line scan in FIG. 6c. FIG. 6c is a spectral domain optical coherence tomography image of the left fundus, through the fovea, showing a thinning of the retina at the fovea (normally over 200 μm) and a loss of retinal lamination, especially centrally (total loss of ellipsoid zone is shown by the black arrow head, and an epiretinal membrane is shown by the white arrows). FIG. 6d is a schematic outline of RPE cell generation from MerTK mutant fibroblasts. Confocal immunofluorescence analysis of MerTK mutant RPE transfected with wild type Dbl3 or Dbl3Y570D is shown in FIG. 6e. RPE cells expressing Dbl3 Y570D undergo efficient recovery of phagocytosis (FIGS. 6e (right panel) and 6g). FIG. 6h illustrates the by-pass mechanism of non-functional MerTK receptors by the engineered phosphomimetic Dbl3 mutant. RPE cells from the MerTK mutant RP individual contain exclusively Dbl plaques or ‘PASPS’ depicted in white with typical complete co-localization as determined by Zeiss 700 software based on the Pearson's Coefficient, whereas cells expressing Dbl3Y570D undergo PASP to cup maturation with characteristic Dbl-POS colocalization at discrete contacts depicted as white rings (FIG. 6i). FIG. 6j shows a schematic illustration of the mechanism of phagocytosis recovery by db3Y570D gene expression. All quantifications are based on n=3 independent experiments and show the data points, means±1SD, the total number of cells analysed for each type of sample across all experiments, and p-values derived from t-tests.

FIG. 7 shows that MRCKβ in the RPE is required for retinal homeostasis. Subretinally injected lentiviral vectors specifically infect the RPE (FIG. 7a; observed as a white band throughout the RPE). Shown is GFP expression upon transduction with a control vector, combined with staining for the RPE marker RPE65 and DNA. MRCKβ expression upon transduction with control CRISPR or two distinct viruses targeting the MRCKβ gene (FIG. 7b). Frozen sections of mouse eyes 21 days after injection with either control or MRCKβ-targeting viruses were stained for RPE65 and DNA (FIG. 7c). MRCKβ knockout leads to a pronounced retinal degeneration.

FIG. 8 shows that MRCKβ is required for membrane remodelling and phagocytosis. siRNA-mediated knockdown of MRCKβ in primary porcine RPE cells lead to prolonged pseudopods and an inhibition of phagocytosis (FIG. 8a to d). Inhibition of MRCKβ kinase activity using BDP5290 results in a membrane remodelling defect and inhibition of phagocytosis in a similar manner to knock-down of MRCKβ (FIG. 8e to h). Note that in both treatments adhesion of POS to RPE cells is unaffected although POS is often misaligned and not centred at extended pseudopods in either MRCKβ siRNA-treated or BDP5290 treated cells as indicated by white arrows in the confocal xy-scanning sections. FIG. 8i shows cortical distribution of MRCKβ in non-polarized THP-1 differentiated macrophages indicating MRCKβ is a highly conserved regulator of the cortical membrane. All quantifications are based on n=3 independent experiments and show the data points, means±1SD, the total number of cells analysed for each type of sample across all experiments, and p-values derived from t-tests.

FIG. 9 shows that POS induced cup formation requires MRCK for centring. Confocal xy scans of porcine primary RPE cells treated with POS for 30 minutes reveal cups encircling the particles with pMLC activity (FIG. 9a; highlighted by white arrows; FIG. 9b). Treatment of cells with the MRCK inhibitor BDP5290 results in un-centred POS around pseudopods that appear not to mature into bona fide cups and lack pMLC activity, indicating a membrane remodelling defect (FIGS. 9c and d). Note that efficiency of POS binding to RPE cells is unaltered as shown in FIG. 3.

FIG. 10 shows that MRCKβ requires myosin-II for cup remodelling. Confocal XY and Z-scans showing inhibition of myosin-II motor activity leads to a cup remodelling defect and reduce efficiency of POS centring (FIG. 10a,b; schematic figure i, ii). Washout of blebbistatin reversed the morphogenic defect of cups and their ability to centre POS, but only if MRCKβ is active (FIG. 10c,d; schematic figure iii, iv).

FIG. 11 shows that POS adhesion to RPE recruits an apical Cdc42 signalling plaque. Confocal Z-scans revealing POS ligation to porcine primary RPE recruits a signalling plaque at nascent pseudopods sites (FIG. 11a to 11d). Note that there is complete co-localization of Cdc42 signalling network with POS at plaques (highlighted by white arrows) whereas in the resultant cup configuration co-localization is only partial at discrete contact sites with the POS (FIGS. 4 and 12). FIG. 11e shows a correlation between size of POS and plaque. FIG. 11f shows that active Cdc42-GTP at PASPs requires Dbl3.

FIG. 12 shows that POS co-localizes with a Cdc42 network at PASPS and cups. Primary porcine RPE cells were treated with POS-FITC for 30 min and analysed by confocal microscopy using Zeiss 700 co-localization software based on the Pearson's correlation coefficient. Co-localization is calculated in grid 3 of the scatter charts. White represents co-localization of POS-FITC with either Dbl (FIG. 12a), Cdc42-GTP (FIG. 12b), MRCKβ (FIG. 12c) or N-WASP (FIG. 12d), which is total in the PASP configuration and only partial, at the particle-contact interface, in the cup configuration. Highlighted outlines of PASPS, cups or POS-cup contacts represent calculation areas of co-localization coefficients, Scale bars: 10 μm.

FIG. 13 shows that pMLC activity increases during PASP to cup remodelling. Confocal sections of primary porcine RPE cells treated with POS-FITC reveal that PASPs display low to moderate pMLC activity (FIG. 13a, b) that increases in the cup configuration (FIG. 13c).

FIG. 14 shows that Dbl3 orchestrates cup morphogenesis and phagocytosis. Confocal xy scanning analysis of ARPE-19 cells stimulated with POS-FITC with or without Dbl3-myc expression (FIGS. 14a and 14b). Note that POS adhesion stimulates Dbl3-dependent cups formation. White arrows indicate cups that are encircling POS. Confocal immunofluorescence analysis of retinal sections from mice injected with control or Dbl3 knockout vectors revealing no significant difference in the overall retinal structure after 7 days but reduced apical F-actin staining in the Dbl3-deficient RPE (FIGS. 14c and 14d). Note that Dbl3 CRISPR experiments were carried out as a set along with MRCKβ CRISPR knock out and therefore reference to the same control data set shown in FIG. 1. Quantifications show means±1SD, n=3, p-values derived from t-tests. siRNA-mediated knock-down of Dbl3 in primary porcine RPE cultures treated with POS for 1 h followed by 2 h chase inhibits phagocytosis as determined by confocal z-sections of porcine primary RPE cells stained with atto647-phalloidin (FIG. 14e).

FIG. 15 shows that N-WASP is required for Dbl3-induced F-actin polymerization. Confocal XY and Z-scanning sections showing ARPE-19 cells expressing Dbl3-myc and stimulated with POS-FITC (highlighted with white single arrows in panels a and b) for 30 minutes require N-WASP for F-actin polymerization during cup formation (FIG. 15a-c). Note, white, grouped arrows highlight loss of Dbl3-myc induced F-actin staining at sites of POS attachment following N-WASP siRNA-mediated knock-down.

FIG. 16 shows that Dbl3-Y570D restores phagocytosis in RP-derived RPE. Autofluorescence image of a magnified area of the posterior pole (FIG. 16a). The speckled darker area at the central macula represents a dropout of RPE cells from an individual carrying a loss of function MerTK mutation that causes retinitis pigmentosa (RP) and vision loss. Confocal XY scanning fluorescence image of RPE cells differentiated from iPSCs from the RP individual reveals an accumulation of PASPs at POS contact sites but no encirclement (FIGS. 16b and 16c). Expression of wildtype Dbl3 causes slight changes to Dbl-POS co-localization but the PASP configuration is maintained (FIG. 16d). Expression of Dbl3Y570D tyrosine phosphomimetic mutant promotes PASP to cup progression despite loss of upstream MerTK function (FIG. 16e). iPSC-derived RPE expressing different forms of Dbl analysed by confocal microscopy using Zeiss 700 co-localization software based on the Pearson's correlation coefficient (FIG. 16f). Note that Dbl3Y570D fully restores progression from complete colocalization in the PASP configuration to discrete contacts that appear as rings in the cup configuration. Schematic illustration of proposed mechanism (FIG. 16g).

FIG. 17 shows that inactivation of the Dbl3 pathway leads to activation of NFκB, which is dependent on IKK, a key upstream regulatory kinase of the NFκB pathway (FIG. 17a). RNA sequencing of human iPSC-derived RPE cells reveals that short-term inactivation of the Dbl3 pathway leads to increased expression of genes activated in inflammation, retinal stress and RPE fibrosis, and decreased expression of genes associated with RPE differentiation and function (FIG. 17b). Confirmation of downregulation of genes required for RPE function upon Dbl3 pathway inactivation by quantitative reverse transcription polymerase chain reaction (FIG. 17c).

FIG. 18 shows that expression of Dbl3 in retinas of Royal College of Surgeons (RCS) rats attenuates loss of retinal function. RCS rats lose vision as they lack functional MerTK, a condition that causes retinitis pigmentosa in humans. Panel A shows sample traces of scotopic electroretinograms (ERGs) after stimulation with 31 Cds/m² recorded 6 weeks after subretinal injection of AAV viruses encoding MerTK, Dbl3 or Dbl3Y529D driven by either CMV or Best1 promoters as indicated. A trace recorded from a healthy Lister Hooded rat is shown as a positive control. Panel B shows that subretinal injection of AAV particles (5×10¹⁰ particles/ml; 2 subretinal injections per eye of 4 μl each) in RCS rats using a vector in which a CMV promoter drives expression of the transgene attenuates loss of vision as measured by recording b-wave amplitudes in electroretinograms (ERG). Measured scotopic b-wave potentials remain significantly higher in injected animals (CMV-Dbl3) than in animals injected with PBS 6-weeks after injection. Positive controls injected with the same type of AAV vectors but expressing MerTK, also retain higher b-wave amplitudes than controls but do not have higher signals than CMV-Dbl3 injected animals (PBS, n=6; CMV-Dbl3, n=5, CMV-MerTK, n=5). Panel C and D show expression of Dbl3 or Dbl3Y570D attenuate loss of retinal function in RCS rats if expressed from the RPE-specific Best1 promoter and delivered as AAV particles (2×10¹¹ particles/ml; 2 subretinal injections per eye of 4 μl each) as assessed by higher recorded b-wave amplitudes in Best1-Dbl3 and Best1-Dbl3Y570D AAV injected eyes comparing to PBS injected controls; Panel C, 6 weeks and Panel D, 12 weeks after injection (PBS, n=8; Best1-Dbl3, n=10, Best1-Dbl3y520D, n=6). Graphs show means+/−1SE; p-values derived from t-tests comparing each AAV-injected group (CMV-Dbl3; CMV-MerTK; Best1-Dbl3; Best1-Dbl3y520D) with PBS control at each light intensity.

FIG. 19 shows that expression of Dbl3 in retinas of RCS rats attenuates degeneration of the retina. Panel A shows confocal scanning images taken from frozen sections of retinas of RCS rats subretinally injected with either PBS or transduced with pAAV-CMV-Dbl3 wt, or pAAV-CMV-MerTK; retinas were analysed 6 weeks after injection (4 eyes were analyzed for each condition). Sections were stained with Phalloidin-Cy5 and Hoechst to detect F-actin and DNA, respectively, in order to delimitate the retinal layers. Panel B shows measurements of the outer nuclear layer (ONL) thickness, a common measure to assess retinal structural integrity. In RCS rats, the ONL thinned to approximately 10 μm. In contrast, transduction of RPE with AAV-CMV-Dbl3 slowed down retinal degeneration as the average thickness of the ONL in these rats was approximately 30 μm. Attenuation of retinal degeneration was also observed after re-introduction of MerTK as previously reported (Smith et al., 2003). Panel C shows confocal scanning images of frozen sections of retinas of RCS rats either injected with PBS (left panel) or AAV particles encoding BEST1-Dbl3 or BEST1-Dbl3Y570D; retinas were analysed 12 weeks after injection (4 eyes were examined for each condition). Panel D shows a quantification of the ONL thickness observed. Structural degeneration of the retina was significantly attenuated upon injection of AAV vectors encoding either wild type or constitutively active (Y570D) Dbl3 as determined by ONL thickness and outer limiting membrane (OLM) integrity. Panel E shows expression of Dbl3 in the RPE of RCS rats transduced with the AAV vectors employed in panels A-D (black arrowheads point to the position of the RPE). Note, dashed lines represent the position of the OLM, which is severely degraded in PBS treated rats. Arrowheads in B and C point to the inner limit of the ONL. Graphs show p-values calculated with Wilcoxon tests. Data points represent measurement of ONL (using Zeiss 2009 Imaging Software) at regular intervals in each retina and several sections from each eye. Sections were cut through the optic nerve point to maintain retinal thickness consistency between treated samples.

DESCRIPTION OF THE SEQUENCES

Protein sequence of human Dbl3 (SEQ ID NO: 1)

| | |
|---|---|
| MQDIAFLSGG RGKDNAWIIT FPENCNFRCI PEEVIAKVLT YLTSIARQNG SDSRFTIILD | 60 |
| RRLDTWSSLK ISLQKISASF PGNLHLVLVL RPTSFLQRTF TDIGFWFSQE DFMLKLPVVM | 120 |
| LSSVSDLLTY IDDKQLTPEL GGTLQYCHSE WIIFRNAIEN FALTVKEMAQ MLQSFGTELA | 180 |
| ETELPDDIPS IEEILAIRAE RYHLLKNDIT AVTKEGKILL TNLEVPDTEG AVSSRLECHR | 240 |
| QISGDWQTIN KLLTQVHDME TAFDGFWEKH QLKMEQYLQL WKFEQDFQQL VTEVEFLLNQ | 300 |
| QAELADVTGT IAQVKQKIKK LENLDENSQE LLSKAQFVIL HGHKLAANHH YALDLICQRC | 360 |
| NELRYLSDIL VNEIKAKRIQ LSRTFKMHKL LQQARQCCDE GECLLANQEI DKFQSKEDAQ | 420 |
| KALQDIENFL EMALPFINYE PETLQYEFDV ILSPELKVQM KTIQLKLENI RSIFENQQAG | 480 |
| FRNLADKHVR PIQFVVPTPE NLVTSGTPFF SSKQGKKTWR QNQSNLKIEV VPDCQEKRSS | 540 |
| GPSSSLDNGN SLDVLKNHVL NELIQTERVY VRELYTVLLG YRAEMDNPEM FDLMPPLLRN | 600 |
| KKDILFGNMA EIYEFHNDIF LSSLENCAHA PERVGPCFLE RKDDFQMYAK YCQNKPRSET | 660 |
| IWRKYSECAF FQECQRKLKH RLRLDSYLLK PVQRITKYQL LLKELLKYSK DCEGSALLKK | 720 |
| ALDAMLDLLK SVNDSMHQIA INGYIGNLNE LGKMIMQGGF SVWIGHKKGA TKMKDLARFK | 780 |
| PMQRHLFLYE KAIVFCKRRV ESGEGSDRYP SYSFKHCWKM DEVGITEYVK GDNRKFEIWY | 840 |
| GEKEEVYIVQ ASNVDVKMTW LKEIRNILLK QQELLTVKKR KQQDQLTERD KFQISLQQND | 900 |
| EKQQGAFIST EETELEHTST VVEVCEAIAS VQAEANTVWT EASQSAEISE EPAEWSSNYF | 960 |
| YPTYDENEEE NRPLMRPVSE MALLY | 985 |

Protein sequence of human Dbl3Y570D (SEQ ID NO: 2)

| | |
|---|---|
| MQDIAFLSGG RGKDNAWIIT FPENCNFRCI PEEVIAKVLT YLTSIARQNG SDSRFTIILD | 60 |
| RRLDTWSSLK ISLQKISASF PGNLHLVLVL RPTSFLQRTF TDIGFWFSQE DFMLKLPVVM | 120 |
| LSSVSDLLTY IDDKQLTPEL GGTLQYCHSE WIIFRNAIEN FALTVKEMAQ MLQSFGTELA | 180 |
| ETELPDDIPS IEEILAIRAE RYHLLKNDIT AVTKEGKILL TNLEVPDTEG AVSSRLECHR | 240 |
| QISGDWQTIN KLLTQVHDME TAFDGFWEKH QLKMEQYLQL WKFEQDFQQL VTEVEFLLNQ | 300 |
| QAELADVTGT IAQVKQKIKK LENLDENSQE LLSKAQFVIL HGHKLAANHH YALDLICQRC | 360 |
| NELRYLSDIL VNEIKAKRIQ LSRTFKMHKL LQQARQCCDE GECLLANQEI DKFQSKEDAQ | 420 |
| KALQDIENFL EMALPFINYE PETLQYEFDV ILSPELKVQM KTIQLKLENI RSIFENQQAG | 480 |
| FRNLADKHVR PIQFVVPTPE NLVTSGTPFF SSKQGKKTWR QNQSNLKIEV VPDCQEKRSS | 540 |
| GPSSSLDNGN SLDVLKNHVL NELIQTERVY VRELYTVLLG YRAEMDNPEM FDLMPPLLRN | 600 |
| KKDILFGNMA EIYEFHNDIF LSSLENCAHA PERVGPCFLE RKDDFQMYAK YCQNKPRSET | 660 |
| IWRKYSECAF FQECQRKLKH RLRLDSYLLK PVQRITKYQL LLKELLKYSK DCEGSALLKK | 720 |
| ALDAMLDLLK SVNDSMHQIA INGYIGNLNE LGKMIMQGGF SVWIGHKKGA TKMKDLARFK | 780 |
| PMQRHLFLYE KAIVFCKRRV ESGEGSDRYP SYSFKHCWKM DEVGITEYVK GDNRKFEIWY | 840 |
| GEKEEVYIVQ ASNVDVKMTW LKEIRNILLK QQELLTVKKR KQQDQLTERD KFQISLQQND | 900 |
| EKQQGAFIST EETELEHTST VVEVCEAIAS VQAEANTVWT EASQSAEISE EPAEWSSNYF | 960 |
| YPTYDENEEE NRPLMRPVSE MALLY | 985 |

Key: Bold = Conserved tyrosine phosphorylation/activation motif
DNA sequence encoding human Dbl3 (SEQ ID NO: 3)

| | |
|---|---|
| cttaatgcaa gacatcgcct tcttgtctgg tggccgggga aaggacaatg cttggatcat | 60 |
| tacgtttcca gaaaactgta attttagatg tataccagag gaagtaatag caaaagtact | 120 |
| tacttacctg acatctattg caaggcaaaa tggatcagac tcccggttta ccattattct | 180 |
| ggatcgaaga ttggatacat ggtcttctct caaaatctct ctccaaaaaa tctcggcttc | 240 |
| cttccctggg aacttgcact tggttttggt tttacgtcct accagctttc ttcaacgaac | 300 |
| gttcacagac attggatttt ggtttagtca ggaggatttt atgcttaaat taccagttgt | 360 |
| tatgctgagc tcagttagtg atttgctgac atacattgat gacaagcaat taacccctga | 420 |
| gttaggcggc accttgcagt actgccacag tgaatggatc atcttcagaa atgctataga | 480 |
| aaattttgcc ctcacagtga aagaaatggc tcagatgtta cagtcctttg gaactgaact | 540 |
| ggctgagaca gaactaccag atgatattcc ctcaatagaa gaaattctgg caattcgtgc | 600 |
| tgaaaggtat catctgttga agaatgatat tacagctgta accaaagaag gaaaaattct | 660 |
| gctaacaaat ctggaagtgc ctgacactga aggagctgtc agttcaagac tagaatgtca | 720 |
| tcggcaaata agtggtgact ggcaaactat taataagttg ctgactcaag tacatgatat | 780 |
| ggaaacagct tttgatggat tttgggaaaa acatcaatta aaaatggagc agtatctgca | 840 |
| actatggaag tttgagcagg attttcaaca gcttgtgact gaagttgaat ttctattaaa | 900 |
| ccaacaagca gaactggctg atgtaacagg gactatagct caagtaaaac aaaaaataaa | 960 |
| aaaattggaa aacttagatg aaaattctca ggagctatta tcaaaggccc agtttgtgat | 1020 |
| attacatgga cacaagcttg cagcaaatca ccattatgca cttgatttaa tctgccagag | 1080 |
| gtgcaatgag ctacgttacc tttctgatat tttggttaat gagataaaag caaaacggat | 1140 |
| acaactcagc aggaccttca aaatgcataa actcctacag caggctcgtc aatgctgtga | 1200 |
| tgaaggggaa tgtcttctag ctaatcagga aatagataag tttcagtcta aagaagatgc | 1260 |
| tcagaaagct ctccaagaca ttgaaaattt tcttgaaatg gctctaccct ttataaatta | 1320 |
| tgaacctgaa acactgcagt atgaatttga tgtaatatta tctcctgagc ttaaggttca | 1380 |
| aatgaagact atacaactca agcttgaaaa cattcgaagt atatttgaga accagcaggc | 1440 |
| tggtttcagg aacctggcag ataagcatgt gaggccaatc caatttgtgg tacccacacc | 1500 |
| tgaaaatttg gtcacatctg ggacaccatt tttttcatct aaacaaggga agaagacttg | 1560 |
| gagacaaaat cagagcaact taaaaattga agtggtgcct gattgtcagg agaagagaag | 1620 |
| ttctggtcca tcctccagtt tggacaatgg caatagcttg gatgttttaa agaaccacgt | 1680 |
| actaaatgaa ctgatacaga ctgagagagt ttatgttcga gaactgtata ctgttttgtt | 1740 |
| gggttataga gcggagatgg ataatccaga gatgtttgat cttatgccac ctctcctgag | 1800 |
| aaataaaaag gacattctct ttggaaacat ggcagaaata tatgaattcc ataacgacat | 1860 |
| tttcttgagc agcctggaaa attgtgctca tgctccagaa agagtgggac cttgtttcct | 1920 |
| ggaaaggaag gatgattttc agatgtatgc aaaatattgt cagaataagc ccagatcaga | 1980 |
| aacaatttgg aggaagtatt cagaatgcgc atttttccag gaatgtcaaa gaaagttaaa | 2040 |
| acacagactt agactggatt cctatttact caaaccagtg caacgaatca ctaaatatca | 2100 |
| gttattgttg aaggagctat taaaatatag caaagactgt gaaggtctgc tctgttgaag | 2160 |
| aaggcactcg atgcaatgct ggatttactg aagtcagtta atgattctat gcatcagatt | 2220 |
| gcaataaatg gctatattgg aaacttaaat gaactgggca agatgataat gcaaggtgga | 2280 |
| ttcagcgttt ggatagggca caagaaaggt gctacaaaaa tgaaggattt ggctagattc | 2340 |
| aaaccaatgc agcgacacct tttcttgtat gaaaaagcca ttgttttttg caaaaggcgt | 2400 |
| gttgaaagtg gagaaggctc tgacagatac ccgtcataca gttttaaaca ctgttggaaa | 2460 |
| atggatgaag ttggaatcac tgaatatgta aaaggtgata accgcaagtt tgaaatctgg | 2520 |
| tatggtgaaa aggaagaagt ttatattgtc caggcttcta atgtagatgt gaagatgacg | 2580 |
| tggctaaaag aaataagaaa tattttgttg aagcagcagg aacttttgac agttaaaaaa | 2640 |
| agaaagcaac aggatcaatt aacagaacgg gataagtttc agatttctct tcagcagaat | 2700 |
| gatgaaaagc aacagggagc ttttataagt actgaggaaa ctgaattgga acacaccagc | 2760 |
| actgtggtgg aggtctgtga ggcaattgcg tcagttcagg cagaagcaaa tacagtttgg | 2820 |
| actgaggcat cacaatctgc agaaatctct gaagaacctg cggaatggtc aagcaactat | 2880 |
| ttctacccta cttatgatga aaatgaagaa gaaaataggc ccctcatgag acctgtgtcg | 2940 |
| gagatggctc tcctatattg a | 2961 |

Key: Bold = start and stop codons
DNA sequence encoding human Dbl3Y570D (SEQ ID NO: 4)

| | |
|---|---|
| cttaatgcaa gacatcgcct tcttgtctgg tggccgggga aaggacaatg cttggatcat | 60 |
| tacgtttcca gaaaactgta attttagatg tataccagag gaagtaatag caaaagtact | 120 |
| tacttacctg acatctattg caaggcaaaa tggatcagac tcccggttta ccattattct | 180 |
| ggatcgaaga ttggatacat ggtcttctct caaaatctct ctccaaaaaa tctcggcttc | 240 |
| cttccctggg aacttgcact tggttttggt tttacgtcct accagctttc ttcaacgaac | 300 |
| gttcacagac attggatttt ggtttagtca ggaggatttt atgcttaaat taccagttgt | 360 |
| tatgctgagc tcagttagtg atttgctgac atacattgat gacaagcaat taacccctga | 420 |
| gttaggcggc accttgcagt actgccacag tgaatggatc atcttcagaa atgctataga | 480 |
| aaattttgcc ctcacagtga aagaaatggc tcagatgtta cagtcctttg gaactgaact | 540 |
| ggctgagaca gaactaccag atgatattcc ctcaatagaa gaaattctgg caattcgtgc | 600 |
| tgaaaggtat catctgttga agaatgatat tacagctgta accaaagaag gaaaaattct | 660 |
| gctaacaaat ctggaagtgc ctgacactga aggagctgtc agttcaagac tagaatgtca | 720 |
| tcggcaaata agtggtgact ggcaaactat taataagttg ctgactcaag tacatgatat | 780 |
| ggaaacagct tttgatggat tttgggaaaa acatcaatta aaaatggagc agtatctgca | 840 |
| actatggaag tttgagcagg attttcaaca gcttgtgact gaagttgaat ttctattaaa | 900 |
| ccaacaagca gaactggctg atgtaacagg gactatagct caagtaaaac aaaaaataaa | 960 |
| aaaattggaa aacttagatg aaaattctca ggagctatta tcaaaggccc agtttgtgat | 1020 |
| attacatgga cacaagcttg cagcaaatca ccattatgca cttgatttaa tctgccagag | 1080 |
| gtgcaatgag ctacgttacc tttctgatat tttggttaat gagataaaag caaaacggat | 1140 |
| acaactcagc aggaccttca aaatgcataa actcctacag caggctcgtc aatgctgtga | 1200 |
| tgaaggggaa tgtcttctag ctaatcagga aatagataag tttcagtcta aagaagatgc | 1260 |
| tcagaaagct ctccaagaca ttgaaaattt tcttgaaatg gctctaccct ttataaatta | 1320 |
| tgaacctgaa acactgcagt atgaatttga tgtaatatta tctcctgagc ttaaggttca | 1380 |
| aatgaagact atacaactca agcttgaaaa cattcgaagt atatttgaga accagcaggc | 1440 |
| tggtttcagg aacctggcag ataagcatgt gaggccaatc caatttgtgg tacccacacc | 1500 |
| tgaaaatttg gtcacatctg ggacaccatt tttttcatct aaacaaggga agaagacttg | 1560 |
| gagacaaaat cagagcaact taaaaattga agtggtgcct gattgtcagg agaagagaag | 1620 |
| ttctggtcca tcctccagtt tggacaatgg caatagcttg gatgttttaa agaaccacgt | 1680 |
| actaaatgaa ctgatacaga ctgagagagt tgatgttcga gaactgtata ctgttttgtt | 1740 |
| gggttataga gcggagatgg ataatccaga gatgtttgat cttatgccac ctctcctgag | 1800 |
| aaataaaaag gacattctct ttggaaacat ggcagaaata tatgaattcc ataacgacat | 1860 |
| tttcttgagc agcctggaaa attgtgctca tgctccagaa agagtgggac cttgtttcct | 1920 |
| ggaaaggaag gatgattttc agatgtatgc aaaatattgt cagaataagc ccagatcaga | 1980 |
| aacaatttgg aggaagtatt cagaatgcgc atttttccag gaatgtcaaa gaaagttaaa | 2040 |
| acacagactt agactggatt cctatttact caaaccagtg caacgaatca ctaaatatca | 2100 |
| gttattgttg aaggagctat taaaatatag caaagactgt gaaggtctgc tctgttgaag | 2160 |
| aaggcactcg atgcaatgct ggatttactg aagtcagtta atgattctat gcatcagatt | 2220 |
| gcaataaatg gctatattgg aaacttaaat gaactgggca agatgataat gcaaggtgga | 2280 |
| ttcagcgttt ggatagggca caagaaaggt gctacaaaaa tgaaggattt ggctagattc | 2340 |
| aaaccaatgc agcgacacct tttcttgtat gaaaaagcca ttgttttttg caaaaggcgt | 2400 |
| gttgaaagtg gagaaggctc tgacagatac ccgtcataca gttttaaaca ctgttggaaa | 2460 |
| atggatgaag ttggaatcac tgaatatgta aaaggtgata accgcaagtt tgaaatctgg | 2520 |
| tatggtgaaa aggaagaagt ttatattgtc caggcttcta atgtagatgt gaagatgacg | 2580 |
| tggctaaaag aaataagaaa tattttgttg aagcagcagg aacttttgac agttaaaaaa | 2640 |
| agaaagcaac aggatcaatt aacagaacgg gataagtttc agatttctct tcagcagaat | 2700 |
| gatgaaaagc aacagggagc ttttataagt actgaggaaa ctgaattgga acacaccagc | 2760 |
| actgtggtgg aggtctgtga ggcaattgcg tcagttcagg cagaagcaaa tacagtttgg | 2820 |
| actgaggcat cacaatctgc agaaatctct gaagaacctg cggaatggtc aagcaactat | 2880 |
| ttctacccta cttatgatga aaatgaagaa gaaaataggc ccctcatgag acctgtgtcg | 2940 |
| gagatggctc tcctatattg a | 2961 |

Key: Bold = start and stop codons, underlined = single point mutation
(Tyrosine to aspartic acid)
RPE65 promoter region (SEQ ID NO: 5)

| | |
|---|---|
| tattgtgcaa ataagtgctc actccaaatt agtggtatat ttattgaagt ttaatattgt | 60 |
| gtttgtgata cagaagtatt tgctttaatt ctaaataaaa attttatgct tttattgctg | 120 |
| gtttaagaag atttggatta tccttgtact ttgaggagaa gtttcttatt tgaaatattt | 180 |
| tggaaacagg tcttttaatg tggaaagata gatattaatc tcctcttcta ttactctcca | 240 |
| agatccaaca aaagtgatta taccccccaa aatatgatgg tagtatetta tactaccatc | 300 |
| attttatagg catagggctc ttagctgcaa ataatggaac taactctaat aaagcagaac | 360 |
| gcaaatattg taaatattag agagctaaca atctctggga tggctaaagg atggagcttg | 420 |
| gaggctaccc agccagtaac aatattccgg gctccactgt tgaatggaga cactacaact | 480 |
| gccttggatg ggcagagata ttatggatgc taagccccag gtgctaccat taggacttct | 540 |
| accactgtcc ctaacgggtg gagcccatca catgcctatg ccctcactgt aaggaaatga | 600 |
| agctactgtt gtatatcttg ggaagcactt ggattaattg ttatacagtt ttgttgaaga | 660 |
| agacccctag ggtaagtagc cataactgca cactaaattt aaaattgtta atgagtttct | 720 |
| caaaaaaaat gttaaggttg ttagctggta tagtatatat cttgcctgtt ttccaaggac | 780 |
| ttctttgggc agtaccttgt ctgtgctggc aagcaactga gacttaatga aagagtattg | 840 |
| gagatatgaa tgaattgatg ctgtatactc tcagagtgcc aaacatatac caatggacaa | 900 |
| gaaggtgagg cagagagcag acaggcatta gtgacaagca aagatatgca gaatttcatt | 960 |
| ctcagcaaat caaaagtcct caacctggtt ggaagaatat tggcactgaa tggtatcaat | 1020 |
| aaggttgcta gagagggtta gaggtgcaca atgtgcttcc ataacatttt atacttctcc | 1080 |
| aatcttagca ctaatcaaac atggttgaat actttgttta ctataactct tacagagtta | 1140 |
| taagatctgt gaagacaggg acagggacaa tacccatctc tgtctggttc ataggtggta | 1200 |
| tgtaatagat atttttaaaa ataagtgagt taatgaatga gggtgagaat gaaggcacag | 1260 |
| aggtattagg gggaggtggg ccccagagaa tggtgccaag gtccagtggg gtgactggga | 1320 |
| tcagctcagg cctgacgctg gccactccca cctagctcct ttctttctaa tctgttctca | 1380 |
| ttctccttgg gaaggattga ggtctctgga aaacagccaa acaactgtta tgggaacagc | 1440 |
| aagcccaaat aaagccaagc atcaggggga tctgagagct gaaagcaact tctgttcccc | 1500 |
| ctccctcagc tgaaggggtg gggaagggct cccaaagcca taactccttt taagggattt | 1560 |
| agaaggcata aaaaggcccc tggctgagaa cttccttctt cattctgcag ttgg | 1614 |

Optimised RPE65 promoter fragment (SEQ ID NO: 6)

| | |
|---|---|
| agatcttcga aatactctca gagtgccaaa catataccaa tggacaagaa ggtgaggcag | 60 |
| agagcagaca ggcattagtg acaagcaaag atatgcagaa tttcattctc agcaaatcaa | 120 |
| aagtcctcaa cctggttgga agaatattgg cactgaatgg tatcaataag gttgctagag | 180 |
| agggttagag gtgcacaatg tgcttccata acattttata cttctccaat cttagcacta | 240 |
| atcaaacatg gttgaatact ttgtttacta taactcttac agagttataa gatctgtgaa | 300 |
| gacagggaca gggacaatac ccatctctgt ctggttcata ggtggtatgt aatagatatt | 360 |
| tttaaaaata agtgagttaa tgaatgaggg tgagaatgaa ggcacagagg tattaggggg | 420 |
| aggtgggccc cagagaatgg tgccaaggtc cagtggggtg actgggatca gctcaggcct | 480 |
| gacgctggcc actcccacct agctcctttc tttctaatct gttctcattc tccttgggaa | 540 |
| ggattgaggt ctctggaaaa cagccaaaca actgttatgg gaacagcaag cccaaataaa | 600 |
| gccaagcatc agggggatct gagagctgaa agcaacttct gttccccctc cctcagctga | 660 |
| aggggtgggg aagggctccc aaagccataa ctccttttaa gggatttaga aggcataaaa | 720 |
| aggcccctgg ctgagaactt ccttcttcat tctgcagttg g | 761 |

Optimised BEST1 promoter fragment (SEQ ID NO: 7)
GAATTCTGTCATTTTACTAGGGTGATGAAATTCCCAAGCAACACCATCCTTTTCAGATAAG
GGCACTGAGGCTGAGAGAGGAGCTGAAACCTACCCGGGGTCACCACACACAGGTGGCAAGG
CTGGGACCAGAAACCAGGACTGTTGACTGCAGCCCGGTATTCATTCTTTCCATAGCCCACA
GGGCTGTCAAAGACCCCAGGGCCTAGTCAGAGGCTCCTCCTTCCTGGAGAGTTCCTGGCAC
AGAAGTTGAAGCTCAGCACAGCCCCCTAACCCCCAACTCTCTCTGCAAGGCCTCAGGGGTC
AGAACACTGGTGGAGCAGATCCTTTAGCCTCTGGATTTTAGGGCCATGGTAGAGGGGGTGT
TGCCCTAAATTCCAGCCCTGGTCTCAGCCCAACACCCTCCAAGAAGAAATTAGAGGGGCCA
TGGCCAGGCTGTGCTAGCCGTTGCTTCTGAGCAGATTACAAGAAGGGACTAAGACAAGGAC
TCCTTTGTGGAGGTCCTGGCTTAGGGAGTCAAGTGACGGCGGCTCAGCACTCACGTGGGCA
GTGCCAGCCTCTAAGAGTGGGCAGGGGCACTGGCCACAGAGTCCCAGGGAGTCCCACCAGC
CTAGTCGCCAGACC
MRCKβ sequence targeted by CRISPR Cas9 (SEQ ID NO: 8)

| | |
|---|---|
| cctggacggg ccgtggcgca ac | 22 |

MRCKβ sequence targeted by CRISPR Cas9 (SEQ ID NO: 9)

| | |
|---|---|
| acaccgagtg cagccactcg g | 21 |

Dbl3 sequence targeted by CRISPR Cas9 (SEQ ID NO: 10)

| | |
|---|---|
| tggagatcga agactggata catgg | 25 |

Sequence in human N-WASP targeted by siRNA (SEQ ID NO: 11)

| | |
|---|---|
| cagauacgac aggguaucca a | 21 |

Sequence in human N-WASP targeted by siRNA (SEQ ID NO: 12)

| | |
|---|---|
| uagagagggu gcucagcuaa a | 21 |

Sequence in porcine Cdc42 targeted by siRNA (SEQ ID NO: 13)

| | |
|---|---|
| gaugaccccu cuacuauug | 19 |

Sequence in porcine Dbl3 targeted by siRNA (SEQ ID NO: 14)

| | |
|---|---|
| aagacaucgc cuuccuguc | 19 |

Sequence in porcine Dbl3 targeted by siRNA (SEQ ID NO: 15)

| | |
|---|---|
| auaccugguc uucucucaa | 19 |

Sequence in porcine MRCKβ targeted by siRNA (SEQ ID NO: 16)

| | |
|---|---|
| cgagaagacu ucgaaauaa | 19 |

Sequence in porcine MRCKβ targeted by siRNA (SEQ ID NO: 17)

| | |
|---|---|
| agagaagacu uugaaauau | 19 |

Conserved Dbl activation motif (SEQ ID NO: 18)

| | |
|---|---|
| TERVYVREL | 9 |

### DETAILED DESCRIPTION

It is to be understood that the terminology used herein is for the purpose of describing particular embodiments of the invention only, and is not intended to be limiting.

In addition, as used in this specification and the appended claims, the singular forms “a”, “an”, and “the” include plural references unless the content clearly dictates otherwise. Thus, for example, reference to “a polynucleotide” includes “polynucleotides”, reference to “a promoter” includes “promoters”, reference to “a vector” includes two or more such vectors, reference to “a molecule” includes two or more such molecules and the like.

All publications, patents and patent applications cited herein are incorporated by reference in their entirety.

The present inventors have identified a conserved apical signalling plaque in the RPE that controls the mechanics of phagocytosis. In particular, the inventors have shown that the RPE forms novel apical signalling plaques (PASPs) in response to POS adhesion to its receptors by recruiting a MerTK tyrosine kinase activated Dbl3/Cdc42/N-WASP/MRCKβ signalling network. The present invention relates to nucleic acid molecules, expression constructs and vectors encoding peptides and proteins that are capable of restoring phagocytosis when introduced into the RPE. According to one embodiment, POS phagocytosis may be restored by expressing exogenous PASP proteins in the RPE.

The terms “nucleic acid molecule” and “polynucleotide” are used interchangeably. Non-limiting examples of polynucleotides include a gene, a gene fragment, messenger RNA (mRNA), complementary DNA (cDNA), genomic DNA, recombinant polynucleotides, plasmids, vectors, expression constructs, nucleic acid probes, and primers. A nucleic acid molecule of the invention may be provided in isolated or purified form. A nucleic acid molecule of the invention may be codon optimised.

The nucleic acid molecule or molecules of the invention encode one or more gene products that are capable of restoring or increasing phagocytosis in the RPE. Gene products that restore or increase phagocytosis in the RPE will reduce one or more of the following phenotypes associated with phagocytosis-related retinal degeneration: thinning of the outer nuclear layer (ONL), reduced numbers of internalised phagosomes, accumulation of extracellular shed photoreceptor fragments, impaired formation of pseudopods, irregular apical membrane and reduced cup retraction, altered RPE structural integrity, altered RPE apical membrane signalling, photoreceptor death, Muller cell activation, Bruch's membrane anomalies, deregulated gene expression and loss of vision.

The skilled person will understand that the term “cup” refers to cup-shaped invaginations of the cell membrane that subsequently close at their distal margins to form phagosomes during phagocytosis. By progression of its rim along a particle surface, this phagocytic cup envelops and eventually encloses the particle by separation of the phagosome membrane from the cell membrane.

Suitable gene products include any protein involved in the MerTK tyrosine kinase activated Dbl3/Cdc42/N-WASP/MRCKβ signalling network. Preferred gene products include Dbl3, N-WASP and MRCKβ polypeptides. In some embodiments, the sequence of the gene product may be identical to the wildtype sequence. In other embodiments, the sequence of the gene product is at least 90%, 91%, 92%, 93%, 94%, 95% 96%, 97%, 98% or 99% identical to the wild type sequence and maintains the function of the wild type sequence. In preferred embodiments, the gene products are modified, constitutively active variants.

In a preferred embodiment, the nucleic acid molecule of the invention encodes a Dbl3 polypeptide or variant thereof that is capable of preventing or improving a phenotype which can be associated with loss of Dbl3 function, or loss of function of a protein involved in the MerTK tyrosine kinase activated Dbl3/Cdc42/N-WASP/MRCKβ signalling network, or with phagocytosis-related retinal degeneration. In a preferred embodiment, the nucleic acid molecule of the invention encodes a Dbl3 polypeptide or variant thereof that maintains the function of the Dbl3 polypeptide. Dbl3 is a Dbl/MCF2 isoform that regulates epithelial morphogenesis, positioning of cell junctions, and the apical-lateral border, as well as apical domain differentiation and size (Zihni et al., 2014). Dbl3 contains an N-terminal Sec-14-like region with an intact Cral-Trio domain that is important for its membrane localization (Zihni et al., 2014). A pleckstrin homology (PH) domain located towards its C-terminal end interacts with the brush border protein ezrin that stabilizes it at the brush border region of epithelia. A Dbl homology (DH) domain situated adjacent to the PH domain is responsible for its Cdc42 GTPase activating potential via phosphorylation that hinder its auto-inhibitory folding conformation.

The Dbl3 polypeptide may be a wildtype Dbl3 protein, such as wildtype human Dbl3 (SEQ ID NO: 1). The Dbl3 nucleic acid molecule may comprise or consist of a wildtype Dbl3 polynucleotide sequence, such as wildtype human Dbl3 (SEQ ID NO: 3).

Dbl3 also drives multiple facets of phagocytosis including actin polymerization, stimulated by N-WASP, and actomyosin contractility, stimulated by MRCKβ. Both N-WASP and MRCKβ are required for phagocytosis; hence, active versions of N-WASP and MRCKβ may be co-expressed to stimulate phagocytosis. Thus nucleic acid molecules of the invention may alternatively or additionally encode N-WASP and MRCKβ.

The Dbl3 polynucleotide sequences may be modified to introduce specific deletions, substitutions or insertions with respect to the native wild-type sequence. According to one embodiment, the modified Dbl3 polypeptide or polynucleotide sequence is at least 90%, 91%, 92%, 93%, 94%, 95% 96%, 97%, 98% or 99% identical to the sequence given in SEQ ID NOs: 1 or 3 respectively, wherein the modified Dbl3 polypeptide or polynucleotide sequence maintains the function of the wild-type Dbl3 polypeptide or polynucleotide sequence. According to one embodiment, the modified Dbl3 polynucleotide sequence is at least 90%, 91%, 92%, 93%, 94%, 95% 96%, 97%, 98% or 99% identical to the sequence given in SEQ ID NO: 3, wherein the modified Dbl3 polynucleotide sequence encodes a protein that maintains the function of the wild-type Dbl3 polypeptide sequence. According to one embodiment, the Dbl3 polypeptide or polynucleotide sequence is at least 90%, 91%, 92%, 93%, 94%, 95% 96%, 97%, 98% or 99% identical to the sequence given in SEQ ID NOs: 1 or 3 respectively, and is capable of preventing or improving one or more of the following phenotypes which can be associated with loss of Dbl3 function or with phagocytosis-related retinal degeneration: thinning of the outer nuclear layer (ONL), reduced numbers of internalised phagosomes, accumulation of extracellular shed photoreceptor fragments, impaired formation of pseudopods, irregular apical membrane, reduced cup retraction, altered RPE structural integrity, altered RPE apical membrane signalling, photoreceptor death, Muller cell activation, Bruch's membrane anomalies, deregulated gene expression, and/or loss of vision.

Dbl3 function can be analysed by any suitable standard technique known to the person skilled in the art or disclosed herein. Means of quantifying POS phagocytosis include quantifying the numbers of phagosomes, quantifying the number of internalised POS, quantifying the accumulation of extracellular shed photoreceptor fragments, or quantifying defects associated with defective phagocytosis such as the thickness of the outer nuclear layer (ONL), or light-induced amplitudes of signals generated electroretinograms.

Dbl3 nucleic acid molecules of the invention include all variants that encode a protein that is capable of preventing or improving a phenotype associated with loss of Dbl3 function. Dbl3 polypeptides of the invention include all variants that are capable of preventing or improving a phenotype associated with loss of Dbl3 function. For example, Dbl3 nucleic acid molecules or polypeptides of the invention include nucleic acid molecules and polypeptides which have been modified to alter the activity of the protein, including modification or deletion of domains associated with autoinhibition.

Dbl3 polypeptides of the invention also include all phosphomimetics, including Dbl3Y570D (e.g. SEQ ID NO: 2). A phosphomimetic is defined as a protein which has been modified to introduce an amino acid substitution that mimics a phosphorylated protein, thereby activating (or deactivating) the protein. Dbl3Y570D, is produced by substituting the tyrosine at amino acid position 570 in SEQ ID NO: 1 with aspartic acid. Tyrosine 570 is located in the DH domain forms part of a conserved TERVYVREL tyrosine phosphorylation motif identified using a bioinformatics NetPhos3.1 algorithm. This motif has been demonstrated to play a key role in the RhoGTPase activating ability of Dbl-homology containing GEFs in response to growth factors (Gupta et al., 2014) and therefore represents a proven activation motif within Dbl3 (SEQ ID NO: 18). Other tyrosine phosphorylation sites, for example, identified using the NetPhos3.1 algorithm, exist in this region that may also potentially represent activation signals for Dbl3.

The nucleic acid molecule or molecules of the invention, for example Dbl3, may be provided in the form of an expression construct, which includes control sequences operably linked to the above sequence, thus allowing for expression of the peptide of the invention in vivo. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. These expression constructs, in turn, are typically provided within vectors (e.g., plasmids or recombinant viral vectors). Such an expression construct may be administered directly to a host subject.

The expression constructs or vectors of the invention have the ability to rescue Dbl3 function and/or POS phagocytosis. “Rescue” generally means preventing or improving one or more of the following phenotypes associated with loss of Dbl3 function: thinning of the outer nuclear layer (ONL), reduced numbers of internalised phagosomes, accumulation of extracellular shed photoreceptor fragments, impaired formation of pseudopods, irregular apical membrane, reduced cup retraction, altered RPE structural integrity, altered RPE apical membrane signalling, photoreceptor death, Muller cell activation, Bruch's membrane anomalies, deregulated gene expression, and/or loss of vision. For example, expression constructs or vectors of the invention have the ability to increase the numbers of internalised phagosomes, reduce or prevent the accumulation of extracellular shed photoreceptor fragments, improve formation of pseudopods, improve the uniformity of the apical membrane and increase cup retraction.

The properties of the expression construct or vector of the invention can also be tested using techniques based on those described in the Examples. In particular, an expression construct of the invention can be assembled into a vector of the invention and delivered to the retina of a Dbl3 or MRCK-deficient test animal, such as a mouse, and the effects observed and compared to a control. Preferably, the control will be the other eye of the same animal, which is either untreated or treated with a control vector such as one containing a reporter gene as opposed to a sequence of the invention.

According to one embodiment, the expression construct comprises a promoter operably linked to a nucleic acid molecule as described above. For example, the promoters of the invention can be used to drive expression of one or more of Dbl3, Cdc42, N-WASP and MRCKβ in the RPE.

Where the expression construct comprises a promoter operably linked to a Dbl3 polynucleotide sequence, the Dbl3 polynucleotide sequence may be a wildtype Dbl3 polynucleotide sequence, or any variant thereof. The Dbl3 polypeptide encoded by the expression construct may comprise the sequence of SEQ ID NO 1, or a sequence that is at least 90%, 91%, 92%, 93%, 94%, 95% 96%, 97%, 98% or 99% identical to the sequence given in SEQ ID NO: 1 that is capable of preventing or improving a phenotype associated with loss of Dbl3 function. According to a preferred embodiment, Dbl3 is a phosphomimetic. Any phosphomimetic that mimics the phosphorylated form of Dbl3 may be used. In particular, the expression construct of the invention may encode Dbl3Y570D (SEQ ID NO: 2).

The promoter may be constitutive or conditionally active. According to one embodiment, the promoter is a CMV promoter. According to other embodiments, the promoter will preferably, but not exclusively, be a RPE-preferred or RPE-specific promoter. RPE-specific expression may be defined as expression that is only present in the RPE, and not in other cell types. RPE-specific expression may be defined as expression that is more than about 10 times greater, 20 times greater, 50 times greater or 100 or more times greater in the RPE than in other cell types, especially photoreceptor cells. Expression in the RPE and other cells types can be measured by any suitable standard technique known to the person skilled in the art. For example, RNA expression levels can be measured by quantitative real-time PCR. Protein expression can be measured by western blotting or immunohistochemistry.

Any RPE-specific gene promoter or fragment thereof may be used to drive expression of the protein of the invention in the RPE. RPE-specific gene promoters and fragments are known in the art and include the promoters of RPE65, BEST1, CRALBP1, TYRP1, and VDM2, or any fragment thereof that retains the ability to express Dbl3 in the RPE. Other RPE-specific promoters include the optimised RPE65 promoter fragment first disclosed in WO 2016/128722.

Where the promoter is an optimised RPE65 promoter, the promoter may comprise a sequence of contiguous nucleotides from SEQ ID NO: 5 that confers RPE-specific expression on an operably linked polynucleotide sequence. The sequence of SEQ ID NO: 5 is 1614 nucleotides in length and does not have RPE-specific activity. Any truncation of SEQ ID NO: 5 that does have RPE-specific activity may be used as the promoter of the invention. Promoter sequences of the invention may for example comprise up to 1500 or 1600 nucleotides of SEQ ID NO: 5 but preferably they contain no more than 1300, no more than 1200, no more than 1100, no more than 1000, no more than 900, no more than 800, no more than 775, no more than 750, no more than 700, no more than 650, no more than 600 or no more than 500 nucleotides of SEQ ID NO: 5. Preferably, sequences of the invention however comprise at least 500, 550, 600, 650, 700, 750, 800, 900, 1000, 1100 or 1200 nucleotides of SEQ ID NO: 5. Preferably, the sequence of the invention is derived from the 3′ end of SEQ ID NO: 5 and includes the 3′ 500, 600, 650, 700, 750, 800, 900, 1000, 1100 or 1200 contiguous nucleotides of SEQ ID NO: 5, or lacks only up to 10, 20, 30, 40, 50, 60, 70, 80, 90, or 100 nucleotides of SEQ ID NO: 5.

Preferred promoters of the invention comprise the sequence of SEQ ID NO: 6 or the sequence of nucleotides 12-761 of SEQ ID NO: 6, typically within a sequence of no more than 800, no more than 850, no more than 900, no more than 1000, no more than 1100 or no more than 1200 contiguous nucleotides of SEQ ID NO: 5. Further preferred promoters comprise at least 750, at least 700, at least 650, at least 600, at least 550 or at least 500 contiguous nucleotides of SEQ ID NO: 6, preferably at least the 500, 550, 600, 650, 700 or 750 nucleotides that are at the 3′ end of SEQ ID NO: 6 or at least the 550, 600, 650, 700 or 750 nucleotides that begin with nucleotide 12 of SEQ ID NO: 6.

Further promoters of the invention are promoters that differ in sequence from the sequences above but retain RPE-specific promoter activity. Such sequences have at least 75%, at least 80%, at least 85%, at least 90%, at least 95%, at least 98% or at least 99% sequence identity to a sequence of contiguous nucleotides from SEQ ID NO: 5 as defined above. Percentage sequence identity of variants is preferably measured over the full length of the corresponding portion of SEQ ID NO: 5, or over a 500, 600, 700, 800, 900, 1000, 1100 or 1200 nucleotide section of SEQ ID NO: 5 aligned with the variant sequence. Sequence identity may be calculated using any suitable algorithm. For example the PILEUP and BLAST algorithms can be used to calculate identity or line up sequences.

A promoter of the invention may also include additional nucleotide sequences not naturally found in the RPE65 promoter region. The promoter sequence of the invention may thus be positioned anywhere within a larger sequence as long as RPE-specific promoter activity is retained. The additional sequence can be 5′ or 3′, or both, to the sequence defined above.

According to one embodiment, the expression construct comprises an RPE-specific promoter comprising: (a) a sequence of contiguous nucleotides from SEQ ID NO: 5 that confers RPE-specific expression on an operably linked polynucleotide sequence, or (b) a sequence having at least 90% sequence identity to said sequence of (a) and that retains RPE-specific promoter activity.

According to one embodiment, the above-mentioned expression construct comprises: (a) no more than 1300 contiguous nucleotides from SEQ ID NO: 5, or (b) a sequence having at least 90% sequence identity to said sequence of no more than 1300 contiguous nucleotides from SEQ ID NO: 5 and retaining RPE-specific promoter activity.

According to one embodiment, the above-mentioned expression construct comprises: (a) no more than 800 contiguous nucleotides from SEQ ID NO: 5, or (b) a sequence having at least 90% sequence identity to said sequence of no more than 800 contiguous nucleotides from SEQ ID NO: 5 and retaining RPE-specific promoter activity.

According to one embodiment, the above-mentioned expression construct comprises:

(a) the sequence of SEQ ID NO: 6 or the sequence of nucleotides 12-761 of SEQ ID NO: 6, or (b) a sequence having at least 90% sequence identity to said sequence of SEQ ID NO: 6, or to nucleotides 12-761 of SEQ ID NO: 6, and retaining RPE-specific promoter activity.

Optionally, said sequence of (a) or (b) in any one of the above-mentioned expression constructs is at least 500 nucleotides in length.

In a preferred embodiment, the promoter of the invention comprises nucleotides 865 to 1614 of the human RPE65 promoter, as described in WO 2016/128722.

Where the promoter is an optimised BEST1 promoter, the promoter may comprise a sequence of contiguous nucleotides from SEQ ID NO: 7 that confers RPE-specific expression on an operably linked polynucleotide sequence. Promoter sequences of the invention may for example comprise all 624 nucleotides of SEQ ID NO: 7, or up to 600 or 500 nucleotides of SEQ ID NO: 7. Preferably, sequences of the invention however comprise at least 400, 450, 500, 550 or 600 nucleotides of SEQ ID NO: 7.

Further promoters of the invention are promoters that differ in sequence from the sequences above but retain RPE-specific promoter activity. Such sequences have at least 75%, at least 80%, at least 85%, at least 90%, at least 95%, at least 98% or at least 99% sequence identity to a sequence of contiguous nucleotides from SEQ ID NO: 7 as defined above. Percentage sequence identity of variants is preferably measured over the full length of the corresponding portion of SEQ ID NO: 7, or over a 400, 450, 500, 600 or more nucleotide section of SEQ ID NO: 7 aligned with the variant sequence. Sequence identity may be calculated using any suitable algorithm. For example the PILEUP and BLAST algorithms can be used to calculate identity or line up sequences.

A promoter of the invention may also include additional nucleotide sequences not naturally found in the BEST1 promoter region. The promoter sequence of the invention may thus be positioned anywhere within a larger sequence as long as RPE-specific promoter activity is retained. The additional sequence can be 5′ or 3′, or both, to the sequence defined above.

According to one embodiment, the expression construct comprises an RPE-specific promoter comprising: (a) a sequence of contiguous nucleotides from SEQ ID NO: 7 that confers RPE-specific expression on an operably linked polynucleotide sequence, or (b) a sequence having at least 90% sequence identity to said sequence of (a) and that retains RPE-specific promoter activity.

According to one embodiment, the above-mentioned expression construct comprises: (a) no more than 600 contiguous nucleotides from SEQ ID NO: 7, or (b) a sequence having at least 90% sequence identity to said sequence of no more than 600 contiguous nucleotides from SEQ ID NO: 7 and retaining RPE-specific promoter activity.

According to one embodiment, the above-mentioned expression construct comprises: (a) at least 400 contiguous nucleotides from SEQ ID NO: 7, or (b) a sequence having at least 90% sequence identity to said sequence of at least 400 contiguous nucleotides from SEQ ID NO: 7 and retaining RPE-specific promoter activity.

According to one embodiment, the above-mentioned expression construct comprises: (a) SEQ ID NO: 7, or (b) a sequence having at least 90% sequence identity to SEQ ID NO: 7 and retaining RPE-specific promoter activity.

Optionally, said sequence of (a) or (b) in any one of the above-mentioned expression constructs is at least 500 nucleotides in length.

One or more other regulatory elements may also be present as well as the promoter. For example, the promoter of the invention can be used in tandem with one or more further promoters or enhancers or locus control regions (LCRs).

Nucleic acid molecules of the invention may be administered by a gene therapy vector, liposome, nanoparticle (for example, a polymeric nanoparticle, solid lipid nanoparticle, or compacted DNA nanoparticle), a dendrimer, polyplex, or polymeric micelles.

When the nucleic acid molecules of the invention are administered by a gene therapy vector, the vector may be of any type, for example it may be a plasmid vector or a minicircle DNA.

Typically, vectors of the invention are viral vectors. The viral vector may be based on the herpes simplex virus, adenovirus or lentivirus. Adeno-associated virus (AAV) vectors or derivatives thereof are particularly attractive as they are generally non-pathogenic; the majority people have been infected with this virus without adverse effects. Furthermore, the immune privilege of ocular tissue renders the eye largely exempt from the adverse immunological responses.

Vectors of the invention typically comprise two inverted terminal repeats (ITRs), preferably one at each end of the genome. An ITR sequence acts in cis to provide a functional origin of replication and allows for integration and excision of the vector from the genome of a cell. The AAV genome typically comprises packaging genes, such as rep and/or cap genes which encode packaging functions for an AAV viral particle. The rep gene encodes one or more of the proteins Rep78, Rep68, Rep52 and Rep40 or variants thereof. The cap gene encodes one or more capsid proteins such as VP1, VP2 and VP3 or variants thereof. These proteins make up the capsid of an AAV viral particle.

The AAV genome may be from any naturally derived serotype or isolate or clade of AAV. The skilled person can select an appropriate serotype, clade, clone or isolate of AAV for use in the present invention on the basis of their common general knowledge. In a preferred embodiment, the serotype is AAV8. Other serotypes may be preferred when targeting cells other than the RPE. For example, AAV2/5 or AAV2.

Preferably the AAV genome will be derivatised for the purpose of administration to patients. Typically, it is possible to truncate the AAV genome significantly to include minimal viral sequence yet retain the above function. Derivatisation reduces the risk of recombination of the vector with wild-type virus, and also to avoid triggering a cellular immune response by the presence of viral gene proteins in the target cell. A derivative may be a chimeric, shuffled or capsid-modified derivative of one or more naturally occurring AAV viruses.

The genome of all AAV serotypes can be enclosed in a number of different capsid proteins. AAV2, for example, can be packaged in its natural AAV2 capsid (AAV2/2) or it can be pseudotyped with other capsids (e.g. AAV2 genome in AAV1 capsid; AAV2/1, AAV2 genome in AAV5 capsid; AAV2/5 and AAV2 genome in AAV8 capsid; AAV2/8). Pseudotyping the AAV2 genome with other AAV capsids can alter cell specificity and the kinetics of transgene expression. For example, when AAV2 is pseudotyped with the AAV4 capsid, transgene expression is targeted specifically to RPE cells (Le Meur et al. 2007).

Chimeric, shuffled or capsid-modified derivatives will be typically selected to provide one or more desired functionalities for the viral vector. Thus, these derivatives may display increased efficiency of gene delivery, decreased immunogenicity (humoral or cellular), an altered tropism range and/or improved targeting of a particular cell type compared to an AAV viral vector comprising a naturally occurring AAV genome, such as that of AAV2. Increased efficiency of gene delivery may be effected by improved receptor or co-receptor binding at the cell surface, improved internalisation, improved trafficking within the cell and into the nucleus, improved uncoating of the viral particle and improved conversion of a single-stranded genome to double-stranded form. Increased efficiency may also relate to an altered tropism range or targeting of a specific cell population, such that the vector dose is not diluted by administration to tissues where it is not needed.

Chimeric capsid proteins include those generated by recombination between two or more capsid coding sequences of naturally occurring AAV serotypes. This may be performed for example by a marker rescue approach in which non-infectious capsid sequences of one serotype are cotransfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins may be generated by engineering the capsid protein sequences to transfer specific capsid protein domains, surface loops or amino acid residues between different capsid proteins. Shuffled capsid proteins may be generated by DNA shuffling or by error-prone PCR.

The sequences of capsid genes may also be genetically modified to introduce specific deletions, substitutions or insertions with respect to the native wild-type sequence. In particular, capsid genes may be modified by the insertion of a sequence of an unrelated protein or peptide within an open reading frame of a capsid coding sequence, or at the N- and/or C-terminus of a capsid coding sequence. The unrelated protein or peptide may advantageously be one which acts as a ligand for a particular cell type, thereby conferring improved binding to a target cell or improving the specificity of targeting of the vector to a particular cell population. The unrelated protein may also be one which assists purification of the viral particle as part of the production process i.e. an epitope or affinity tag. The site of insertion will typically be selected so as not to interfere with other functions of the viral particle e.g. internalisation, trafficking of the viral particle. The skilled person can identify suitable sites for insertion based on their common general knowledge.

The invention additionally encompasses the provision of sequences of an AAV genome in a different order and configuration to that of a native AAV genome. The invention also encompasses the replacement of one or more AAV sequences or genes with sequences from another virus or with chimeric genes composed of sequences from more than one virus. Such chimeric genes may be composed of sequences from two or more related viral proteins of different viral species.

AAV viruses are replication incompetent. Therefore helper virus functions, preferably adenovirus helper functions, will typically also be provided on one or more additional constructs to allow for AAV replication.

For the avoidance of doubt, the invention also provides an AAV viral particle comprising a vector of the invention. The AAV particles of the invention include transcapsidated forms wherein an AAV genome or derivative having an ITR of one serotype is packaged in the capsid of a different serotype. The AAV particles of the invention also include mosaic forms wherein a mixture of unmodified capsid proteins from two or more different serotypes makes up the viral envelope. The AAV particle also includes chemically modified forms bearing ligands adsorbed to the capsid surface. For example, such ligands may include antibodies for targeting a particular cell surface receptor.

The invention additionally provides a host cell comprising a vector or AAV viral particle of the invention.

The vector of the invention may be prepared by standard means known in the art for provision of vectors for gene therapy. Thus, well established public domain transfection, packaging and purification methods can be used to prepare a suitable vector preparation.

A particularly preferred packaged viral vector for use in the invention comprises an AAV8 serotype or a derivatised genome of AAV2 in combination with AAV5 or AAV8 capsid proteins.

All of the above additional constructs may be provided as plasmids or other episomal elements in the host cell, or alternatively one or more constructs may be integrated into the genome of the host cell.

### Exogenous Proteins

Alternatively, POS phagocytosis may be restored by delivery of exogenous components of the Dbl3/Cdc42/N-WASP/MRCKβ signalling network to the RPE. For example, one or more of Dbl3, MRCKβ or N-WASP may be delivered to the RPE. According to one embodiment, active variants of one or more of Dbl3, MRCKβ or N-WASP may be delivered to the RPE. Means of delivering protein therapeutics to cells are known in the art. For example, proteins of the invention may be conjugated to peptide which aid delivery to the RPE. Peptides of the invention, such as Dbl3, may be conjugated to a cell penetrating peptide (CPPs). CPPs of the invention aid the uptake of substances into tissues. CPPs of the invention aid the intracellular uptake of substances. CPPs of the invention enhance the endocytosis of substances. Intracellular uptake and endocytosis can be measured by the skilled person by methods known in the art, for example as described in Davis et al (2014).

### Small Molecules

Also provided are small molecules that bind components of the Dbl3/Cdc42/N-WASP/MRCKβ signalling network and stimulate their activity. For example, provided are small molecules which may be administered to the RPE to activate Dbl3, MRCKβ or N-WASP. Also provided are small molecules which may be administered to rescue POS phagocytosis and, thereby, attenuate retinal degeneration. Small molecules may be delivered to the RPE using liposomes, CPPs, intravitreal injection, topical eye drops or combination thereof.

### Pharmaceutical Compositions

The invention provides a pharmaceutical composition comprising a nucleic acid molecule, expression construct, vector, protein or small molecule of the invention. The composition may additionally comprise a pharmaceutically acceptable carrier, diluent, excipient, adjuvant, buffer, stabiliser, and/or other materials well known to those skilled in the art. Such materials should be non-toxic and should not interfere with the efficacy of the active ingredient.

The precise nature of the carrier or other material may be determined by the skilled person according to the route of administration; for example, direct retinal, subretinal or intravitreal injection.

The pharmaceutical composition is typically in liquid form. Liquid pharmaceutical compositions generally include a liquid carrier such as water, petroleum, animal or vegetable oils, mineral oil or synthetic oil. Physiological saline solution, magnesium chloride, dextrose or other saccharide solution or glycols such as ethylene glycol, propylene glycol or polyethylene glycol may be included. In some cases, a surfactant, such as pluronic acid (PF68) 0.001% may be used.

For injection at the site of affliction, the active ingredient will be in the form of an aqueous solution which is pyrogen-free and has suitable pH, isotonicity and stability. Those skilled in the art will be able to prepare suitable solutions using, for example, isotonic vehicles such as Sodium Chloride Injection, Ringer's Injection, Lactated Ringer's Injection, Hartmann's solution. Preservatives, stabilisers, buffers, antioxidants and/or other additives may be included, as required.

For delayed release, the vector may be included in a pharmaceutical composition which is formulated for slow release, such as in microcapsules formed from biocompatible polymers or in liposomal carrier systems according to methods known in the art.

Dosages and dosage regimes can be determined within the normal skill of the medical practitioner responsible for administration of the composition. The dosage of active agent(s) may vary, depending on the reason for use, the individual subject, and the mode of administration. The dosage may be adjusted based on the subject's weight, the age and health of the subject, and tolerance for the compound(s) or composition.

### Host Cells

The invention additionally provides a host cell comprising a nucleic acid, such as a vector or a viral vector, or AAV viral particle disclosed herein. Any suitable host cell can be used to produce the nucleic acids, as such vectors, disclosed herein. In general, such cells will be transfected mammalian cells, but other cell types, e.g. insect cells, can also be used. In terms of mammalian cell production systems, HEK293 and HEK293T are preferred for AAV vectors. BHK or CHO cells may also be used.

### Methods of Therapy and Medical Uses

Nucleic acid molecules, expression constructs, vectors, proteins, small molecules and compositions of the invention can be used to treat and/or prevent retinal disorders or dystrophies. Retinal disorders or dystrophies can be defined as diseases of the retina, characterised by progressive loss of photoreceptor cells and concomitant loss of vision. Many retinal disorders or dystrophies are also characterised by defective phagocytosis of POS. Phenotypes associated with phagocytosis-related retinal degeneration include thinning of the outer nuclear layer (ONL), reduced numbers of internalised phagosomes, accumulation of extracellular shed photoreceptor fragments, impaired formation of pseudopods, irregular apical membrane and reduced cup retraction, altered RPE structural integrity, altered RPE apical membrane signalling, photoreceptor death, Muller cell activation, Bruch's membrane anomalies, deregulated gene expression and loss of vision.

The retinal disorders or dystrophies may be inherited retinal disorders or dystrophies, such as retinitis pigmentosa or macular degeneration. The macular degeneration may be age-related macular degeneration (AMD), for example wet or neovascular AMD or geographic atrophy, or inherited dystrophies such as Stargardt and Sorsby diseases.

Reduced phagocytic activity is also thought to contribute to increased intraocular pressure and glaucoma, as well as to neuroinflammatory diseases in the brain. Phagocytic cells in the brain also use MerTK to bind ligand and activate phagocytosis. Therefore, vectors or expression constructs comprising sequences encoding components of the signalling pathway described above (i.e. Dbl3, Cdc42, N-WASP or MRCKβ) and a promoter may be used to target ocular and non-ocular tissues other than the RPE.

The terms “patient” and “subject” may be used interchangeably. The patient is preferably a mammal. The mammal may be a commercially farmed animal, such as a horse, a cow, a sheep or a pig, a laboratory animal, such as a mouse or a rat, or a pet, such as a cat, a dog, a rabbit or a guinea pig. The patient is more preferably human. The subject may be male or female. The subject is preferably identified as being at risk of, or having, one of the above-mentioned retinal disorders or dystrophies.

The terms “treat,” “treated,” “treating,” or “treatment” as used herein refer to both therapeutic treatment and prophylactic or preventative measures, wherein the object is to prevent or slow down (lessen) an undesired physiological condition, disorder or disease, or to obtain beneficial or desired clinical results. Beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects.

For the purposes of this invention, beneficial or desired clinical results include increased numbers of internalised phagosomes, reduced accumulation of extracellular shed photoreceptor fragments, increased/improved formation of pseudopods, increased/improved cup retraction, normal RPE structural integrity, normal RPE apical membrane signalling, reduced photoreceptor death, reduced Muller cell activation, reduced Bruch's membrane anomalies, and reduced loss of vision, when compared to the same patient prior to treatment.

The patient may be asymptomatic and/or may have a predisposition to the disease. As such, the invention also provides a method or use that comprises a step of identifying whether or not a subject is at risk of developing, or has, one of the above-mentioned retinal disorders.

A prophylactically effective amount of nucleic acid molecules, expression constructs, vectors, proteins, small molecules or compositions may be administered to a subject. A prophylactically effective amount is an amount which prevents the onset of one or more symptoms of the disease. As such, in some embodiments, nucleic acid molecules, expression constructs, vectors, proteins, small molecules or compositions may be administered in order to prevent or delay the onset of one or more symptoms of retinal disorders, including, but not limited to retinitis pigmentosa or macular degeneration.

Alternatively, nucleic acid molecules, expression constructs, vectors, proteins, small molecules or compositions may be administered once the symptoms of the disease have appeared in a subject. A therapeutically effective amount of the nucleic acid, expression construct, vector, protein small molecule or composition may be administered to a subject. As used herein, “therapeutically effective amount” means an amount of a nucleic acid set forth herein that, when administered to a mammal, is effective in producing the desired therapeutic effect.

Provided is a nucleic acid molecule, expression construct, vector, protein, small molecule or composition for use in a method for treating or preventing retinal disorders or dystrophies. For example, provided is an expression construct comprising a RPE-specific or RPE-preferred promoter operably linked to a nucleic acid sequence encoding a protein that is capable of increasing POS phagocytosis for use in a method for treating or preventing retinal disorders or dystrophies. According to one embodiment, the RPE-specific or RPE-preferred promoter is operably linked to a nucleic acid sequence encoding Dbl3.

Also provided is an AAV vector comprising the above-mentioned expression construct for use in a method for treating or preventing retinal disorders or dystrophies.

Also provided is Dbl3 or a variant thereof that is capable of preventing or improving a phenotype associated with loss of Dbl3 function, for use in a method for treating or preventing retinal disorders or dystrophies in a patient in need thereof.

Provided is a method for treating or preventing retinal disorders or dystrophies in a patient in need thereof, said method comprising administering a therapeutically effective amount of the above-mentioned nucleic acid molecule, expression construct, vector, protein, small molecule or composition to the patient. For example, provided is a method for treating or preventing retinal disorders or dystrophies in a patient in need thereof, said method comprising administering to a patient a therapeutically effective amount of an expression construct comprising a RPE-specific or RPE-preferred promoter operably linked to a nucleic acid sequence encoding a protein that is capable of increasing POS phagocytosis. According to one embodiment, the RPE-specific or RPE-preferred promoter is operably linked to a nucleic acid sequence encoding Dbl3.

Also provided is a method for treating or preventing retinal disorders or dystrophies in a patient in need thereof, said method comprising administering to a patient a therapeutically effective amount of a AAV vector comprising the above-mentioned expression construct.

Also provided is a method for treating or preventing retinal disorders or dystrophies in a patient in need thereof, said method comprising administering to a patient Dbl3, or a variant thereof that is capable of preventing or improving a phenotype associated with loss of Dbl3 function.

Provided is the use of the above-mentioned nucleic acid molecule, expression construct, vector, protein, small molecule or composition in a method for treating or preventing retinal disorders or dystrophies in a patient in need thereof. For example, provided is the use of a therapeutically effective amount of an expression construct comprising a RPE-specific or RPE-preferred promoter operably linked to a nucleic acid sequence encoding a protein that is capable of increasing POS phagocytosis in a method for treating or preventing retinal disorders or dystrophies in a patient in need thereof. According to one embodiment, the RPE-specific or RPE-preferred promoter is operably linked to a nucleic acid sequence encoding Dbl3.

Also provided is the use of an AAV vector comprising the above-mentioned expression construct in a method for treating or preventing retinal disorders or dystrophies in a patient in need thereof.

Also provided is the use of Dbl3, or a variant thereof that is capable of preventing or improving a phenotype associated with loss of Dbl3 function, in a method for treating or preventing retinal disorders or dystrophies in a patient in need thereof.

The invention also provides the use of a nucleic acid, expression construct, vector or protein or small molecule in the manufacture of a medicament for the treatment or prevention of retinal disorders or dystrophies, such as retinitis pigmentosa or macular degeneration. For example, provided is the use of a therapeutically effective amount of an expression construct comprising a RPE-specific or RPE-preferred promoter operably linked to a nucleic acid sequence encoding a protein that is capable of increasing POS phagocytosis in the manufacture of a medicament for treating or preventing retinal disorders or dystrophies in a patient in need thereof. According to one embodiment, the RPE-specific or RPE-preferred promoter is operably linked to a nucleic acid sequence encoding Dbl3.

Also provided is the use of an AAV vector comprising the above-mentioned expression construct in the manufacture of a medicament for treating or preventing retinal disorders or dystrophies in a patient in need thereof.

Also provided is the use of Dbl3, or a variant thereof that is capable of preventing or improving a phenotype associated with loss of Dbl3 function, in the manufacture of a medicament for treating or preventing retinal disorders or dystrophies in a patient in need thereof.

The nucleic acid molecules, expression constructs, vectors, proteins, small molecules and compositions may be administered to the patient by direct retinal, subretinal or intravitreal injection. This includes direct delivery to RPE cells. The nucleic acid molecules, expression constructs, vectors, proteins, small molecules and compositions may specifically target RPE cells without entering any other cell populations.

The dose of the nucleic acid molecules, expression constructs, vectors, proteins, small molecules and compositions as disclosed herein may be determined according to various parameters, especially according to the age, weight and condition of the patient to be treated, the route of administration, and the required regimen. A physician will be able to determine the required route of administration and dosage for any particular patient.

A typical single dose is between 10¹⁰ and 10¹² genome particles, depending on the amount of remaining retinal tissue that requires transduction. A genome particle is defined herein as an AAV capsid that contains a single stranded DNA molecule that can be quantified with a sequence specific method (such as real-time PCR). That dose may be provided as a single dose, but may be repeated for the fellow eye or in cases where the nucleic acid, such as a vector, as disclosed herein may not have targeted the correct region of retina for whatever reason (such as surgical complication). The treatment is preferably a single permanent treatment for each eye, but repeat injections, for example in future years and/or with different AAV serotypes may be considered.

The nucleic acid molecules, expression constructs, vectors, proteins, small molecules and compositions disclosed herein can be used in combination with any other therapy for the treatment and/or prevention of retinal disorders or retinal dystrophies, including retinitis pigmentosa or macular degeneration. In particular, the nucleic acid molecules, expression constructs, vectors, proteins, small molecules and compositions disclosed herein can be used in combination with any other therapy for the treatment and/or prevention of age-related macular degeneration (AMD), for example wet or neovascular AMD or geographic atrophy, or inherited dystrophies such as Stargardt and Sorsby diseases.

The nucleic acid molecules, expression constructs, vectors, proteins, small molecules and compositions disclosed herein can be packaged into a kit.

The following Examples illustrate the invention.

### EXAMPLES

### Example 1: Materials and Methods

### Cell Culture and Generation of Mammalian Expression and Viral Vectors

Human ARPE-19 cells were obtained from ATCC. Primary porcine RPE cells were isolated from pig eyes as described in Tsapara, A. et al. ARPE-19 and porcine RPE cells were grown in DMEM supplemented with 10% foetal bovine serum. Once isolated porcine RPE were confluent they were cultured in DMEM medium containing 1% FBS and passaged no more than 2 times. For phagocytosis assays, the cells were transferred to medium containing 2.5% foetal bovine serum. Fresh batches of ARPE-19 cells from a contamination-free stock that had been tested for mycoplasma (MycoAlert; Promega Inc.) were used every 6 to 8 weeks. Cells were then weekly stained with Hoechst dye to reveal nuclei and DNA of contaminants such as mycoplasma. iPSC cells carrying inactivating mutations in MerTK were characterised previously and were differentiated into RPE cells as described in Ramsden, C. M. et al. MRCK inhibitor BDP5290 was generously provided by Michael Olson (Cancer Research UK Beatson Institute, Glasgow, UK) and was synthesized by the Cancer Research UK Beatson Institute Drug Discovery Group. It was used at a concentration of 10 mM. Blebbistatin (used at 10 μM) and Wiskostatin (used at 5 μM) were purchased from Tocris Bioscience. The expression plasmid for Dbl3-myc was as described in Zihni, C. et al (2014).

Dbl3Y570D and Dbl3Y570F substitutions were introduced into Dbl3-myc by PCR using the CloneAmp HiFi PCR premix and In-Fusion cloning (Clontech Inc.). pAAV-CMV-Dbl3, pAAV-CMV-Dbl3Y570D, pAAV-BEST1-Dbl3, pAAV-BEST1-Dbl3Y570D were all constructed by using a pAAV-CMV-Lac Z vector backbone. Briefly, for pAAV-CMV-Dbl3 constructs Lac-Z region was removed and replaced with Dbl3 or Dbl3 Y570D using In-Fusion (Clontech) cloning protocols. To make pAAV-BEST1-Dbl3, the CMV promotor region was removed and replaced by the BEST1 promoter, which was generated by PCR using genomic DNA as a template. Recombinant AAV serotype 8 was then produced for subretinal injections using analogous methods previously described for other serotypes (Smith et al., 2003). Constructs were verified by sequencing.

CRISPR Cas9 lentiviral vectors were obtained from Sigma-Aldrich and were constructed in pLV-U6g-EPCG. The sequences targeted were for MRCKβ 5′-CCTGGACGGGCCGTGGCGCAAC-3′ (SEQ ID NO: 7) and 5′-ACACCGAGTGCAGCCACTCGG-3′ (SEQ ID NO: 8), and for Dbl3 5′-TGGAGATCGAAGACTGGATACATGG-3′ (SEQ ID NO: 9). VSV-G pseudotyped lentiviral particles were generated using 293T cells.

Plasmids were transfected using TransIT-X2 (Cambridge Bioscience).

### RCS Rats, Subretinal Injections, ERGs, Immunohistochemistry

All animal experiments were conducted in accordance with the policies on the Use of Animals and Humans in Neuroscience Research and an approved personal project licence from the Home Office UK. Royal College of Surgeons RCS rats were used for this study. RCS rats carry a genetic defect in the gene encoding MerTK, which disrupts phagocytosis and leads to blindness. Control electroretinograms (ERGs) were acquired from wild-type Lister Hooded rats. Subretinal injections were performed on postnatal day 10. Surgery was performed under direct ophthalmoscopic control through an operating microscope. The tip of a 1.5 cm, 34-gauge hypo-dermic needle (Hamilton, Switzerland) was inserted tangentially through the sclera of the rat eye, causing a self-sealing wound tunnel. The needle tip was brought into focus in the subretinal space and approximately 4 μl of viral suspension were injected to produce a bullous retinal detachment in the superior and inferior hemisphere around the injection sites. ERGs were recorded in a standardized manner by the same investigators at time points up to 12 weeks after treatment. All animals were dark-adapted overnight. The rats were anaesthetized with an intraperitoneal injection of ketamine (60 mg/kg) and xylazine (8 mg/kg). The pupils were dilated using phenylephrine 2.5% and tropicamide 1% to each eye. A drop of 2% hydroxy-propyl-methyl-cellulose was placed on each cornea to keep it moisturized. ERG measurements were performed with a Diagnosys Celeris rodent ERG apparatus using mouse stimulators for rats up to 7 weeks of age and rat stimulators for animals older than 7 weeks. Light stimulation and measurements were controlled with Espion software using light intensities from 0.005 to 50 Cds/m². At given time points, rats were culled and eyes were enucleated, fixed in 3% paraformaldehyde for 60 minutes, washed with PBS and then embedded in sucrose followed by OCT for the preparation of cryosections. Frozen sections were then thawed and permeabilized using 0.5% Triton-X/0.1% Saponin/PBS (TSP) and then blocked with 0.1% TSP with 1% donkey serum in PBS for 1 hour. Primary antibodies were incubated in blocking solution overnight. After washing with blocking solution and incubation with secondary fluorescently tagged antibodies, the sections were washed again and then mounted using Prolong Gold. Confocal microscopic analysis was carried out using a Zeiss 700 and Zen imaging software.

### Transfection

Cells were cultured and transfected with Lipofectamine RNAiMAX (Thermo Fisher Scientific) or Vitromer Blue (Lipocalyx) using siRNAs targeting the following sequences: human N-WASP 5′-CAGAUACGACAGGGUAUCCAA-3′ (SEQ ID NO: 11) and 5′-UAGAGAGGGUGCUCAGCUAAA-3′ (SEQ ID NO: 12); porcine Cdc42, 5′-GAUGACCCCUCUACUAUUG-3′ (SEQ ID NO: 13); porcine Dbl3 5′-AAGACAUCGCCUUCCUGUC-3′(SEQ ID NO: 14) and 5′-AUACCUGGUCUUCUCUCAA-3′ (SEQ ID NO: 15); and porcine MRCKβ 5-CGAGAAGACUUCGAAAUAA-3′ (SEQ ID NO: 16) and 5′-AGAGAAGACUUUGAAAUAU-3′ (SEQ ID NO: 17). The non-targeting control siRNAs and those for human Cdc42, MRCKβ, and Dbl3 were as described previously in Zihni, C. et al. (2016) and Zihni, C. et al. (2017).

### Mice Maintenance

Wild-type mice (C57BL/6J) were purchased from Harlan Laboratories (Blackthorn, UK). Additional rd1 mice were purchased from Charles River (Margate, UK). All mice were maintained under cyclic light (12 h light-dark) conditions; cage illumination was 7 foot-candles during the light cycle. All experiments were approved by the local Institutional Animal Care and Use Committees (UCL, London, UK) and conformed to the guidelines on the care and use of animals adopted by the Society for Neuroscience and the Association for Research in Vision and Ophthalmology (Rockville, Md., USA).

### Generation of CRISPR Lentiviral Particles and Subretinal Injection

CRISPR Cas9 lentiviral vectors were obtained from Sigma-Aldrich and were constructed in pLV-U6g-EPCG. The sequences targeted were for MRCKβ 5′-CCTGGACGGGCCGTGGCGCAAC-3′ (SEQ ID NO: 8) and 5′-ACACCGAGTGCAGCCACTCGG-3′ (SEQ ID NO: 9), and for Dbl3 5′-TGGAGATCGAAGACTGGATACATGG-3′ (SEQ ID NO: 10). VSV-G pseudotyped lentiviral particles were generated using 293T cells (Tsapara, A et al (2010). Subretinal injections were performed under direct retinoscopy thorough an operating microscope. The tip of a 1.5-cm, 34-gauge hypodermic needle (Hamilton) was inserted tangentially through the sclera of the mouse eye, causing a self-sealing wound tunnel. The needle tip was brought into focus between the retina and retinal pigment. Animals received double injections of 2 μl each to produce bullous retinal detachments in the superior and inferior hemisphere around the injection sites. Eyes were assigned as treated and (contralateral) control eyes using a randomization software.

### Mammalian Antibodies and Immunological Methods

Fixation and processing cells and mouse tissue sections was as previously described in Zihni, C. et al (2017). The following antibodies were used: RPE65 mouse monoclonal (Millipore) 1/300 for immuno-fluorescence; p-MLC S19, mouse monoclonal (Cell Signaling Technology) immunofluorescence 1/100 and immunoblotting 1/1000; Dbl(3), rabbit polyclonal (Santa Cruz Biotechnology) immunofluorescence 1/200; MRCKβ, rabbit polyclonal (Santa Cruz Biotechnology) immunofluorescence 1/200 and immunoblotting 1/500; anti-Cdc42-GTP mouse monoclonal (NewEast Biosciences) immunofluorescence 1/50; N-WASP, rabbit polyclonal (Santa Cruz Biotechnology) immunofluorescence and immunoblotting 1/1000; MerTK rabbit monoclonal (Abcam 52968) immunofluorescence 1/100; Integrin αVβ5, mouse monoclonal (Clone P1F6) (Abcam 24694) immunofluorescence 1/100; pFAK Y397, rabbit polyclonal (Invitrogen) immunofluorescence 1/100; pSrc Y416, rabbit polyclonal (Cell Signaling Technology) immunofluorescence 1/100; Phalloidin-Atto 647 reagent was obtained from Sigma and diluted 1/1000. Affinity-purified and cross-adsorbed Alexa488-, Cy3- and Cy5-labelled donkey anti-mouse, rabbit, or goat secondary antibodies were from Jackson ImmunoResearch Laboratories (1/300 diluted from 50% glycerol stocks). Affinity-purified HRP-conjugated goat anti mouse and rabbit, and donkey anti goat secondary antibodies 1/5000 were also from Jackson ImmunoResearch Laboratories (1/5000 diluted from 50% glycerol stocks). For immunofluorescence analysis cells and tissues were mounted using Prolong Gold antifade reagent (Life technologies) and imaging was performed using Zeiss 700 and 710 confocal microscopes and a 64× oil lens/NA1.4. Images were processed using Zeiss Zen 2009 and Adobe Photoshop CS5 and 10 software. Co-immunoprecipitation and immunoblotting were carried out using methods previously described and were repeated at least three times.

### Isolation of Photoreceptor Outer Segments

Pig's eyes were obtained from a slaughter house. Briefly in chilled and sterile conditions, each eye was cut into two halves (cornea, including 5 mm into the sclera). After removing the lens the eyeball was filled with PBS and neural retina removed using forceps. The PBS was then replaced with 10× Trypsin and incubated at 37° C. for 30 mins. The trypsin was pipetted several times to dislodge the RPE cells, and cell suspensions were centrifuged at ambient temperature at 800 rpm for 5 mins and washed with PBS. The cells were plated into 6-well tissue culture plates with DMEM containing 10% FBS and antibiotics.

### Electron Microscopy

All steps were performed at room temperature as described in Zihni, C. et al (2014) and Zihni, C. et al (2017). Briefly, cell monolayers were fixed in a mixture of 3% (vol/vol) glutaraldehyde and 1% (wt/vol) PFA in 0.08 M sodium cacodylate buffer (CB), pH 7.4, for 2 h at room temperature and left overnight at 4° C. Before osmication, the primary fixative solution was replaced by a 0.08 M cacodylate buffered solution of 2.5% glutaraldehyde and 0.5% (wt/vol) tannic acid. After two brief rinses in CB, specimens were osmicated for 2 h in 1% (wt/vol) aqueous osmium tetroxide, dehydrated by 10-min incubations in 50%, 70%, 90%, and three times 100% ethanol. semithin sections (0.75 μm) for light microscopy and ultrathin sections (50-70 nm) for electron microscopy were cut from sawed out blocks with diamond knives (Diatome; Leica). Semithin sections were stained with 1% toluidine blue/borax mixture at 60° C. and ultrathin sections were stained with Reynold's lead citrate. Stained ultrathin sections were examined in a transmission electron microscopy (1010; JEOL) operating at 80 kV and images were recorded using an Onus B digital camera and DigitalMicrograph (Gatan, Inc.)

### Statistical Analysis

Photoreceptor outer segments (POS) internalized into the cell body was measured in Zeiss 700 confocal Z-sections by analysing FITC-labelled POS particles using Zeiss Zen2000 imaging software or by analysing Gold-labelled POS by transmission electron microscopy. Microvillar organization was measured in electron micrographs using Image-J software. Sheets of MV comprising 0, 0-5 or greater than 5, width of MV at nM intervals were measures as units every 1.5 μM image width. Immunostaining pixel intensity was measured using Image J software. For each measurement background was measured and subtracted from the sample value. Co-localization coefficients were measured in Zeiss confocal EY-scans using Zeiss Zen 2000 co-localization software that applies the principal of the Pearson's coefficient to selected subcellular structures and using cross hairs function to subtract background from each channel. Co-localization coefficients at specifically PASPs, total cups or POS-signalling plaque contacts at cups were measured by selecting these areas, indicated by coloured outlines. For the quantifications shown, the provided n values refer to independent experiments and the numbers added in brackets in the graphs refer to the total of analysed cells. Statistical significance was tested in most experiments using two-tailed Student's t-tests with an n value of 3, or as indicated in Figure legends. Wilcoxon tests also were used where indicated.

### Example 2: MRCKβ is Required for RPE Function In Vivo

Cdc42 signalling components that control cytoskeletal remodelling during apical epithelial polarization continue to localize apically in mature, post-mitotic epithelial cells in vivo. The Cdc42 effector MRCKβ, in particular, displays a high level of conservation across species for regulating apical actomyosin contractility. Hence, the inventors asked whether such components play a role in RPE phagocytosis.

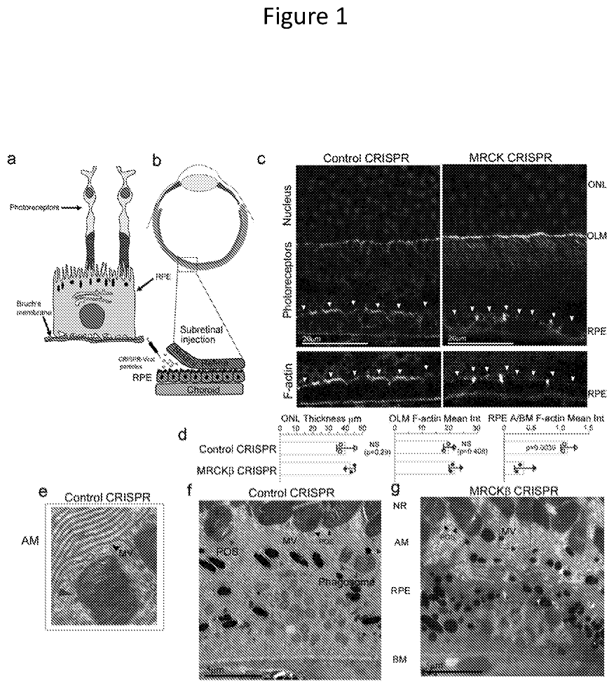

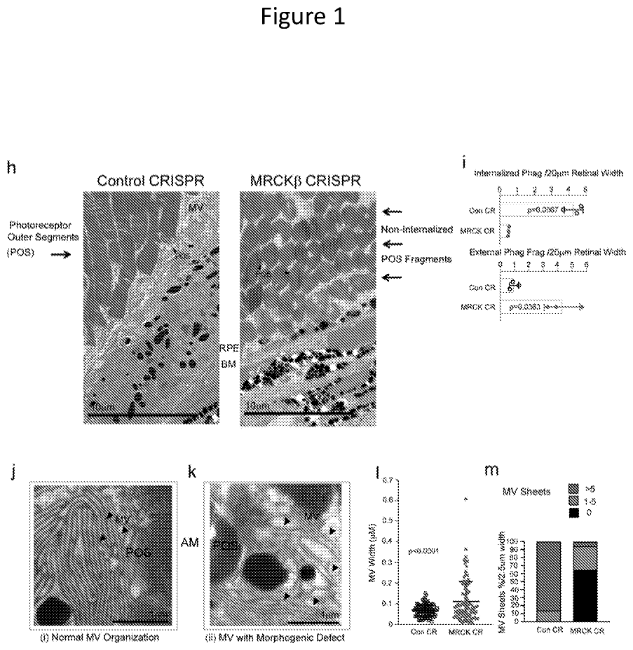

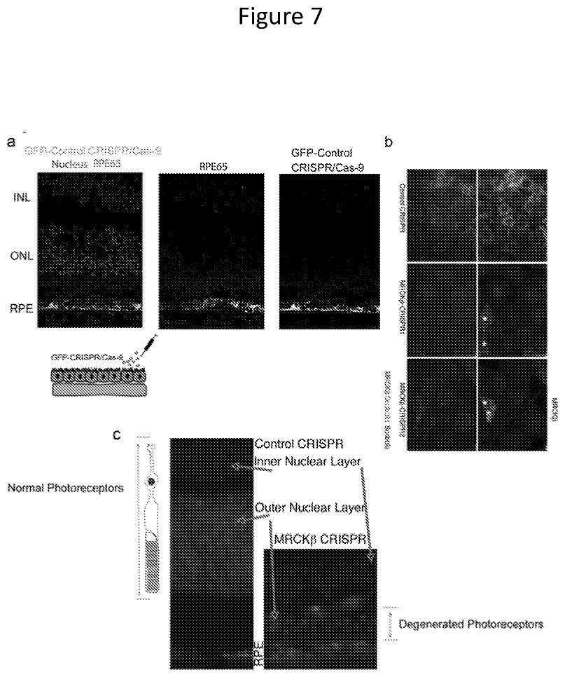

To test this, the inventors injected lentiviral CRISPR-Cas9 control or knockout vectors targeting MRCKβ into the subretinal space of mice, which results in specific transduction of the RPE (FIG. 1a-d; FIG. 7a,b). Adult animals were used as their RPE is non-proliferative and fully polarized.

Analysis of retinal tissue sections by confocal microscopy revealed that mice maintained for 21 days post-injection displayed a striking thinning of the outer nuclear layer (ONL), a hallmark of advanced retinal degeneration (FIG. 7c). If mice were analysed after 7 days, the ONL thickness and the outer limiting membrane F-actin intensity were unchanged (FIG. 1c,d), indicating that the general architecture and integrity of the retina were maintained. However, MRCKβ knockout RPE cells displayed a reduced apical-basal F-actin intensity ratio (FIG. 1c,d), indicating that the apical membrane cytoskeleton is affected.

The apical RPE membrane is covered by specialized, extended microvilli that function in phagocytosis; hence, the inventors investigated the ultrastructure of the RPE using transmission electron microscopy (TEM).

In control RPE cells, the apical microvilli were ordered and packed into linear arrays that extended to and around photoreceptor outer segments (POS) as previously reported in Bonilha, V. L. et al. (2006)(FIGS. 1e,f). Cells also contained internalized phagosomes indicating functional RPE (FIG. 1e,f).

The apical microvilli of MRCKβ knockout RPE appeared disordered, and, unlike control RPE, microvilli in contact with extracellular POS appeared unorganised (FIG. 1g, j,k). Measurement of microvilli packing (sheet integrity) and thickness, to quantify order, revealed a decrease in microvilli sheets and uneven distribution of individual microvilli thickness order (FIG. 1j-m). Strikingly, MRCKβ-targeted RPE displayed a reduction in internalized phagosomes and an accumulation of extracellular shed photoreceptor fragments (FIGS. 1h,i).

Thus, MRCKβ is required in adult RPE apical membrane organization, POS phagocytosis and retinal integrity.

### Example 3: MRCKβ is Essential for POS-Induced Phagocytic Membrane Remodelling

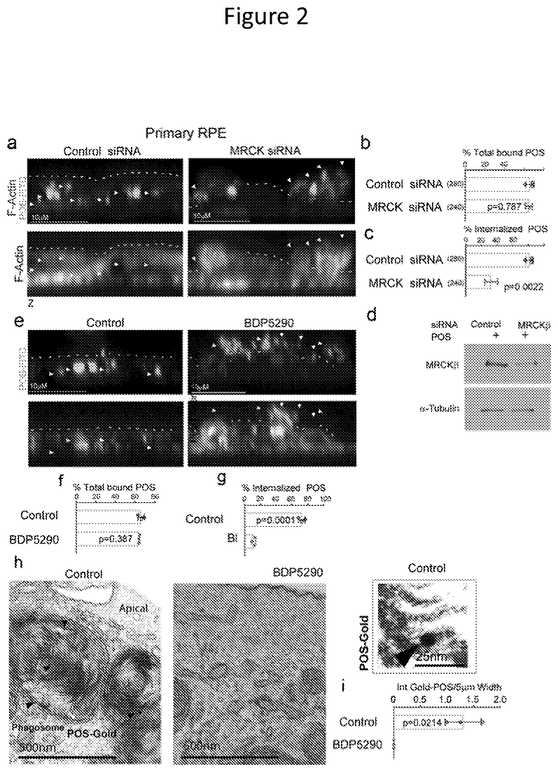

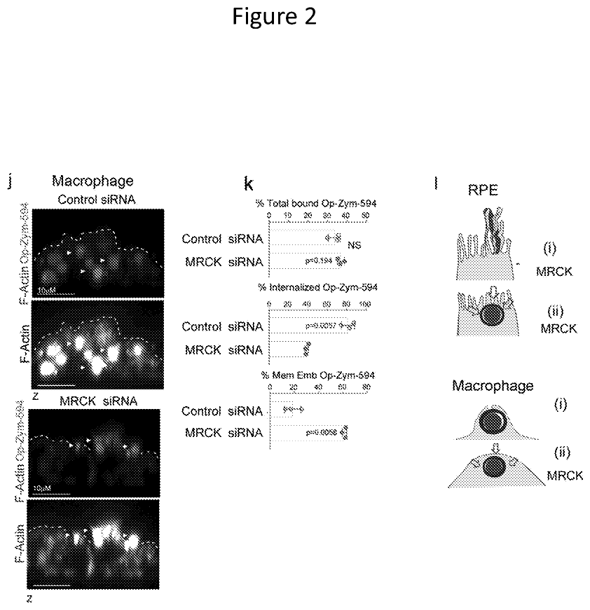

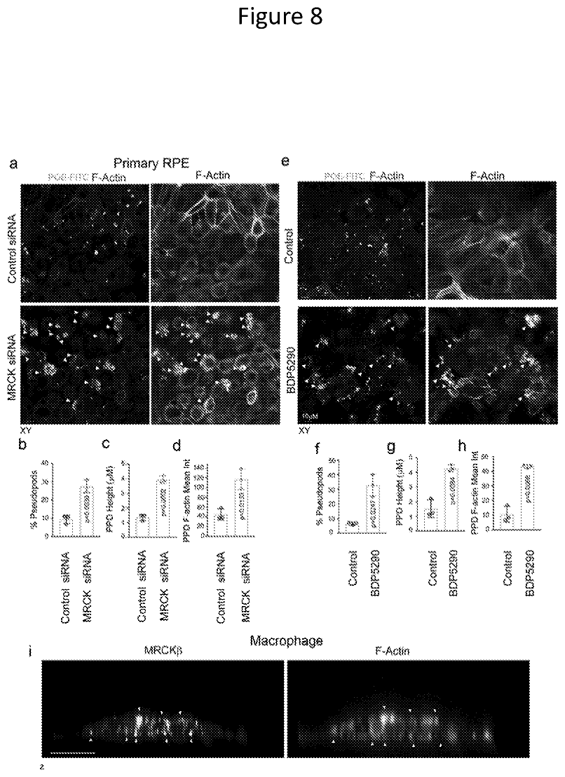

The inventors next asked whether MRCKβ is directly involved in POS internalization using porcine primary RPE cells. Cells were exposed to POS-FITC for 1 h followed by a 2 h chase in the presence or absence of an inhibitor of MRCKβ BDP5290, or following siRNA-mediated MRCKβ knockdown. Analysis of confocal z-sections revealed that siRNA silencing or inhibition of kinase activity strongly inhibited the ability of RPE cells to internalize POS (FIGS. 2a-1 and 9a-d). MRCK was not required for POS to attach to the RPE, indicating integrin αVβ5-POS binding was not affected (FIGS. 2b and 2f). POS binding stimulates the formation of extended pseudopods that retract with internalization of POS. MRCKβ inactivation resulted in an irregular apical membrane with elongated F-actin positive pseudopod structures upon POS binding, that were more numerous with greater F-actin intensity (FIGS. 2a and e, right panels, and 8a-h). Since MRCKβ activates myosin II motors, these results are in agreement with previous reports that myosin II-dependent contractility functions to limit F-actin polymerization by promoting depolymerization; a process that's necessary for cup retraction during particle internalization. Furthermore, the prolonged pseudopods failed to encircle POS and the particles were often misaligned from the centre of the pseudopods (FIGS. 8a and 8e). Ultrastructure analysis of RPE treated with GOLD-labelled POS using TEM microscopy confirmed inhibition of phagocytosis (FIG. 2h,i). Thus, MRCKβ controls POS-induced membrane remodelling during phagocytic cup formation and particle internalization.

Macrophages are the best studied phagocytic cell type. Their cell surface organization and mechanism of phagocytic cargo engulfment is considerably different to RPE. In Fc-receptor mediated phagocytosis, macrophages undergo actin independent membrane remodelling as a first step in the path to internalize particles and the role of myosin-II is receptor-dependent. Therefore, macrophages are a good model to determine whether the importance of MRCK in phagocytosis is conserved. As expected, in macrophages differentiated from THP-1 cells MRCKβ is not polarized, unlike RPE, but also associates with the cell cortex (FIG. 8i; arrows highlight non-polarized cortical distribution of MRCKβ in THP-1 differentiated macrophages). Fluorescent IgG-Opsonized Zymosan-yeast particles, which engage the Fc-receptor and stimulate Cdc42 activation, were efficiently internalized by control siRNA-treated cells but not cells transfected with MRCKβ siRNA (FIG. 2j,k). MRCKβ depletion led to increased accumulation of particles in the F-actin rich cortex of the cell membrane. This phenotype is reminiscent to what has been reported for myosin-II inactivation in macrophages, the physiological substrate of MRCKβ, which leads to inhibition of secession from cortical actin and membrane fusion.

Thus, MRCKβ-driven membrane remodelling is required for phagocytosis in both RPE and a non-epithelial cell type. However, while RPE cells require MRCKβ for cup formation and internalization, macrophages require the kinase only for actomyosin-dependent internalization (FIG. 2l).

### Example 4: Cooperation of MRCKβ and N-WASP

To understand the function of MRCKβ in membrane remodelling in response to POS adhesion, the inventors analysed its activity and function at earlier time points.

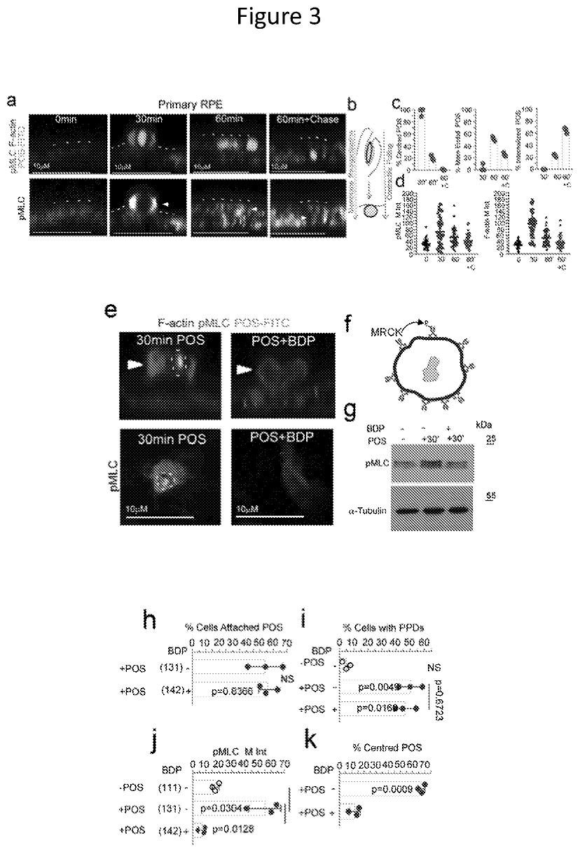

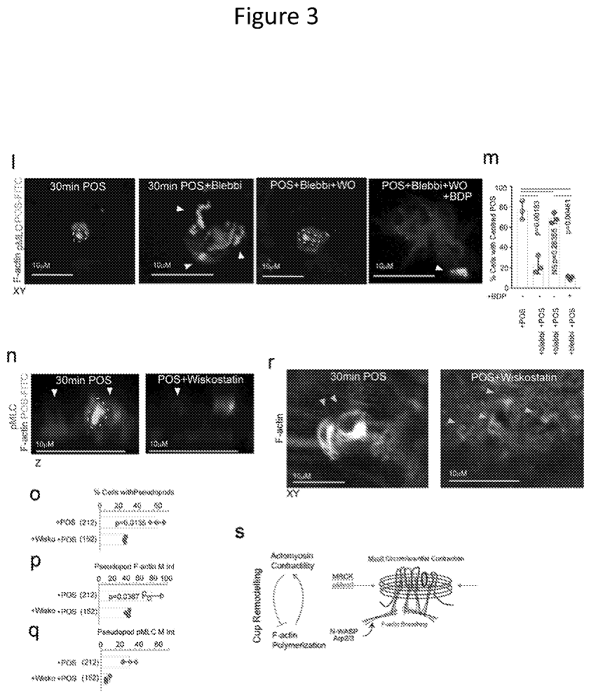

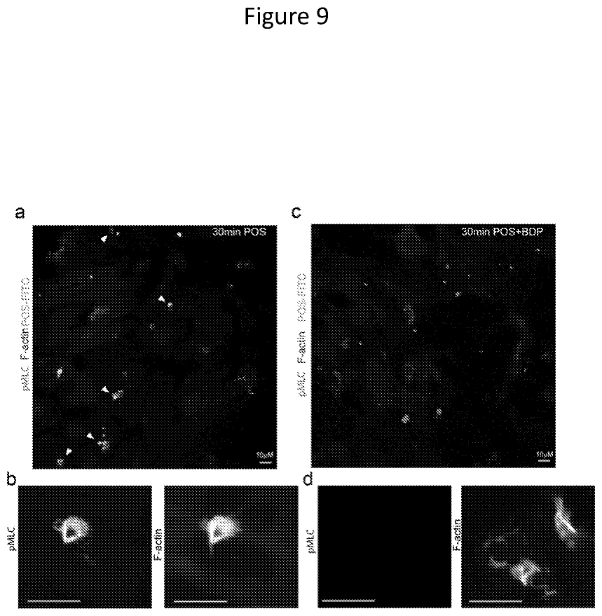

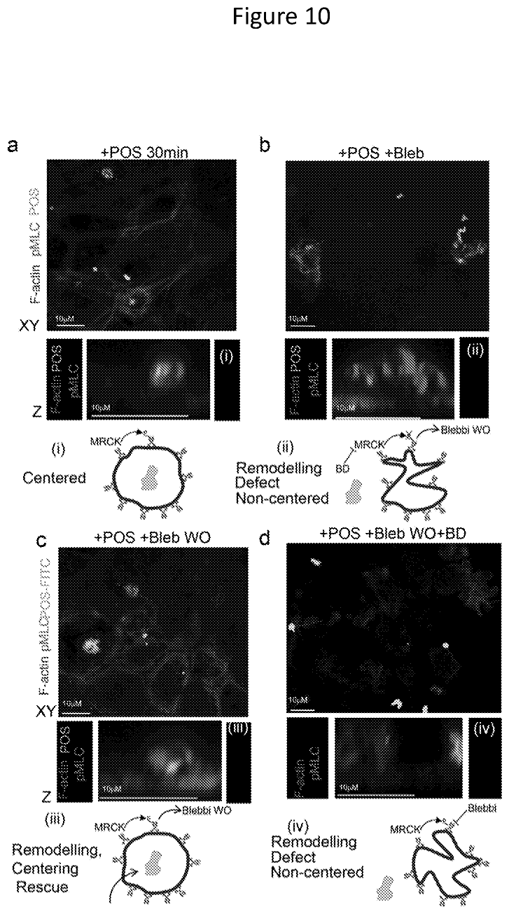

Maximal myosin light chain (MLC) phosphorylation occurred at 30 minutes, colocalising with extended pseudopods encircling the POS (FIGS. 3a-i and 10a-b). Inhibition of MRCK activity abrogated MLC phosphorylation at forming cups; however, pseudopods still formed but POS failed to localize at their centre (FIG. 3e,i,k). No defect in POS binding was observed (FIGS. 3h and 9a,c).

The inventors asked whether MRCKβ is responsible for myosin-II activation. Reversible myosin-II inhibition using blebbistatin resulted in disorganised pseudopods and loss of efficient POS centring, similar to MRCK inhibition (FIGS. 3l,m and 10a-d). Washout of blebbistatin rapidly restored normal cup organization and POS centring in the absence but not in the presence of the MRCK inhibitor, which also led to inhibition of MLC phosphorylation. Thus, myosin-II motor activity at cups, remodelling of the cup and centring of POS are dependent on MRCK activity.

F-actin polymerization is necessary for myosin motor activity. Therefore, the inventors asked whether actin polymerisation is also regulated by Cdc42. N-WASP plays an important role in actin nucleation and polymerization by activating ARP2/3. Inhibition of N-WASP using Wiskostatin resulted in a drastic reduction in the number of cells forming normal pseudopods (FIG. 3o). Nevertheless, pseudopod formation at a reduced level was still observed, possibly reflecting actin polymerization stimulated by the previously described integrin αvβ5-Rac pathway. However, these pseudopods contained less F-actin (approx. 50%) when compared to control cells, and pMLC staining was reduced, indicating reduced myosin-II activity (FIG. 3n-q).

Thus, two Cdc42 effectors, N-WASP and MRCKβ are required for POS-induced membrane remodelling, contractile cup assembly with centred POS and phagocytosis.

### Example 5: Dbl3/Cdc42 Orchestrates De Novo Cup Formation

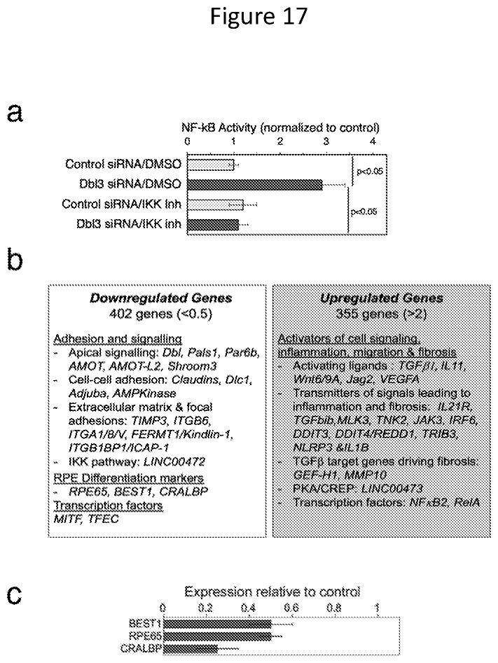

Inactivation of the Dbl3 pathway leads to activation of NFκB, which is dependent on IKK, a key upstream regulatory kinase of the NFκB pathway (FIG. 17a). RNA sequencing of human iPSC-derived RPE cells reveals that short-term inactivation of the Dbl3 pathway leads to increased expression of genes activated in inflammation, retinal stress and RPE fibrosis, and decreased expression of genes associated with RPE differentiation and function (FIG. 17b,c).

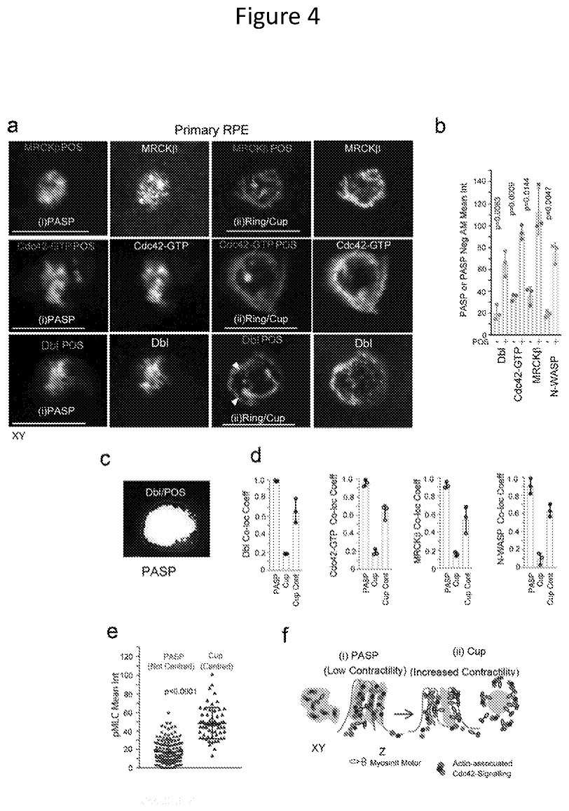

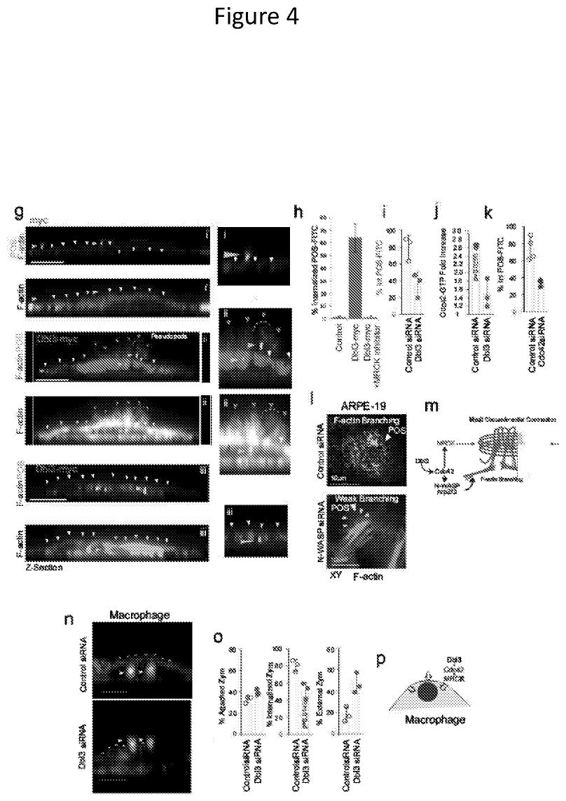

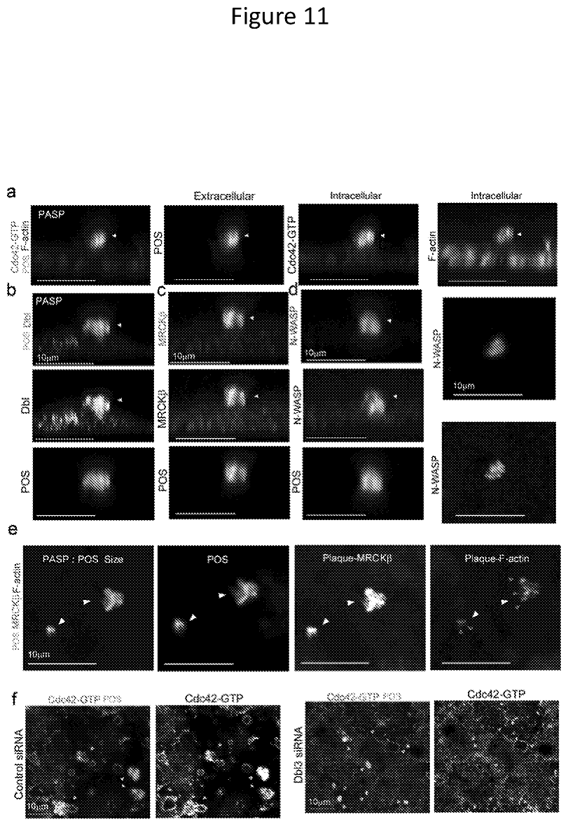

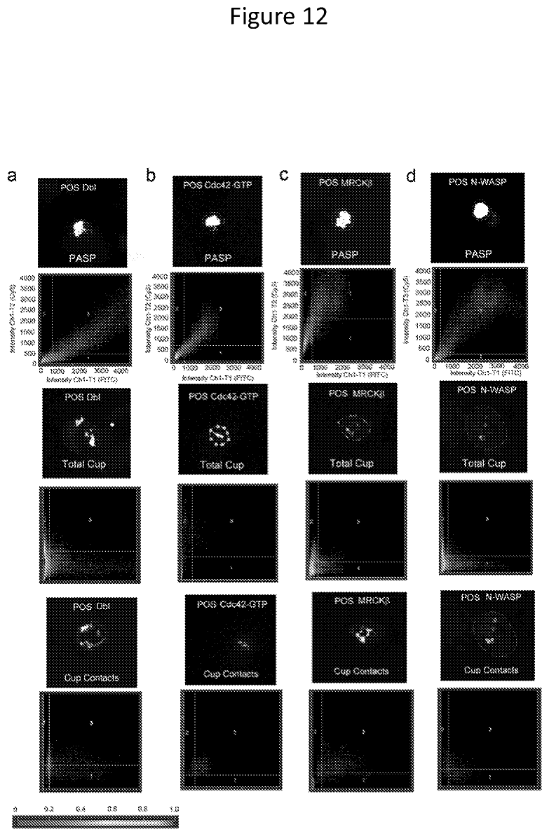

Analysis of Cdc42 signalling components in porcine primary RPE cells incubated with POS for 30 min revealed two populations of POS adhesion-associated structures that contained GTP-bound Cdc42, Dbl, MRCKβ and N-WASP (FIGS. 4a and 11a-c). The first population displayed complete co-localization of these components with POS (FIGS. 4a-f and 11a-c). These structures are referred to as POS-Associated Signalling Plaques (PASP) since the recruited Cdc42 signalling machinery drives cytoskeletal remodelling. The second structure, which in a time course followed plaque assembly, were the cup structures that appear as rings in the xy-sections.

At cups the Cdc42 signalling components only co-localized with the centred particle at the POS-cup interface and not the centre, unlike PASPs where co-localization was almost total (FIGS. 4a-d and 12a-d). The PASP represents a concentrated cytoskeletal regulatory machinery that relays the extracellular signal coming from the POS to remodel the cortex and membrane to induce phagocytosis.

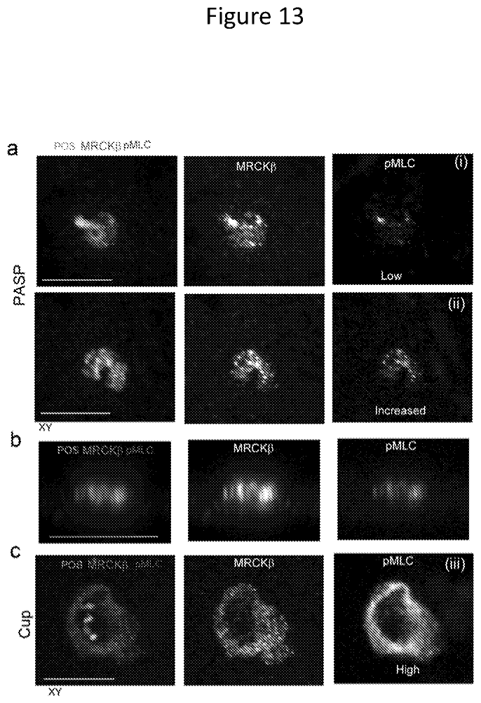

Measurement of MLC phosphorylation as an indicator of actomyosin contractility revealed a lower activity at PASPs and a twofold increase at cups (FIGS. 4e and 13a-c), supporting the importance of actomyosin contractility in cup morphogenesis during PASP to cup conversion and POS centring.

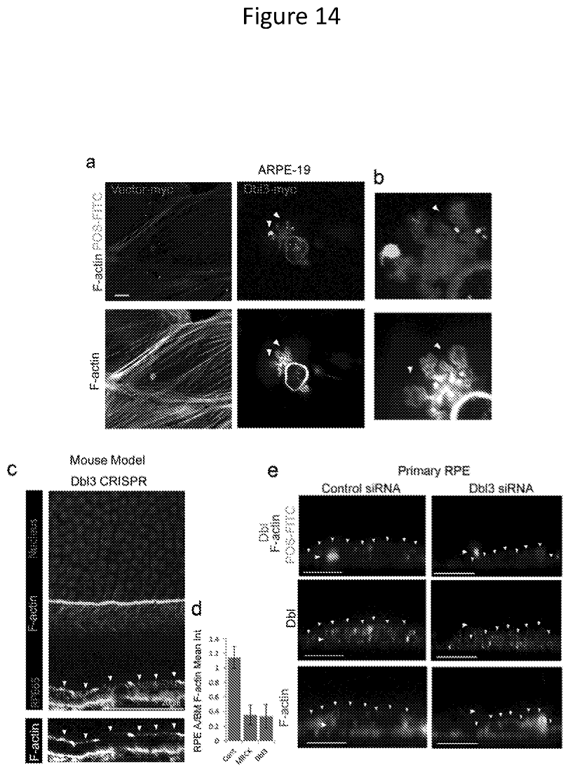

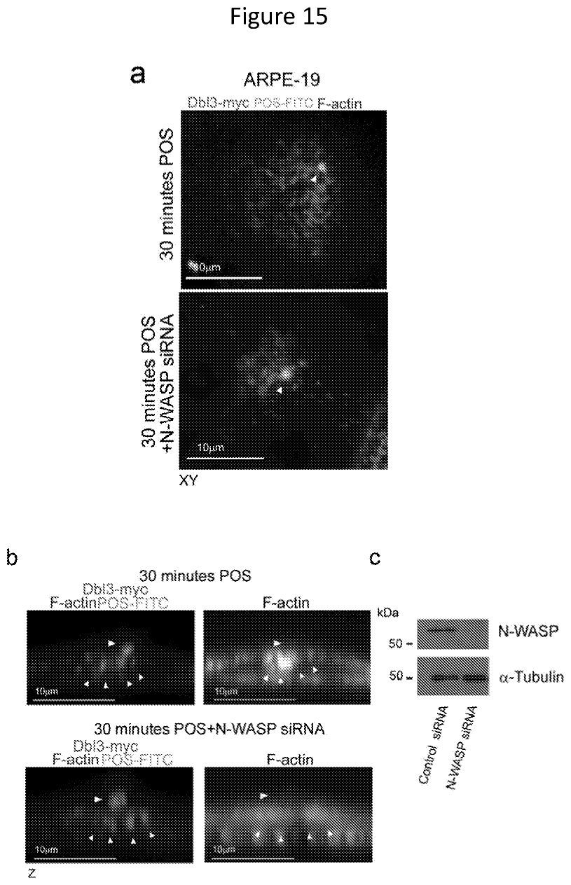

To determine whether the Cdc42 GEF Dbl3 is a driver of phagocytosis, the inventors used ARPE-19 cells, which normally phagocytose very slowly, as a gain of function model system. Exposure of these cells to POS-FITC for 1 h followed by a 2 h chase resulted in efficient association of POS-FITC to the apical membrane although particle internalization was extremely inefficient, indicating slow phagocytosis (FIG. 4g, panels labelled (i)). In contrast, exogenous expression of Dbl3-myc led to POS-dependent induction of prominent pseudopods and phagocytic cups, and POS internalization (FIGS. 4g, panels labelled (ii) and (iii), and 14a,b). Dbl3-myc localized along these membrane protrusions (FIG. 4g—highlighted by dashes; FIG. 14a—highlighted by arrows).

Conversely, siRNA-mediated knock down of Dbl3 in porcine RPE resulted in inhibition of POS internalization (FIG. 4i; FIG. 14e) and, in vivo, CRISPR-mediated knockout of Dbl3 resulted in a redistribution of F-actin in the RPE as observed for the MRCKβ knockout (7 days after injection) (FIG. 14c,d).

Knockdown and inhibition experiments in ARPE-19 cells and porcine primary RPE cells further indicate that Dbl3-induced phagocytosis depends on Cdc42, N-WASP and MRCKβ and that POS-Dbl3-induced remodelling of the F-actin cytoskeleton during plaque assembly requires N-WASP (FIGS. 4h-m and 15a-b). Knock-down of Dbl3 in macrophages displayed an inhibitory effect on phagocytosis (FIG. 4n-p) similar to MRCKβ (FIG. 2j-1) indicating that Dbl3-MRCK signalling is highly conserved in phagocytic membrane remodelling.

Thus, POS-membrane adhesion stimulates recruitment of a Cdc42 signalling network to form a cytoskeletal signalling plaque that drives membrane remodelling and contractile cup formation. The GEF Dbl3 plays a central role in this signalling network, stimulating Cdc42 and, thereby, MRCKβ and N-WASP effector mechanisms.

### Example 6: Dbl3 is Activated by MerTK-Gas-6

The inventors next asked whether the POS receptors integrin αVβ5 and MerTK, and their associated effectors FAK and c-Src are part of the PASP signalling network.

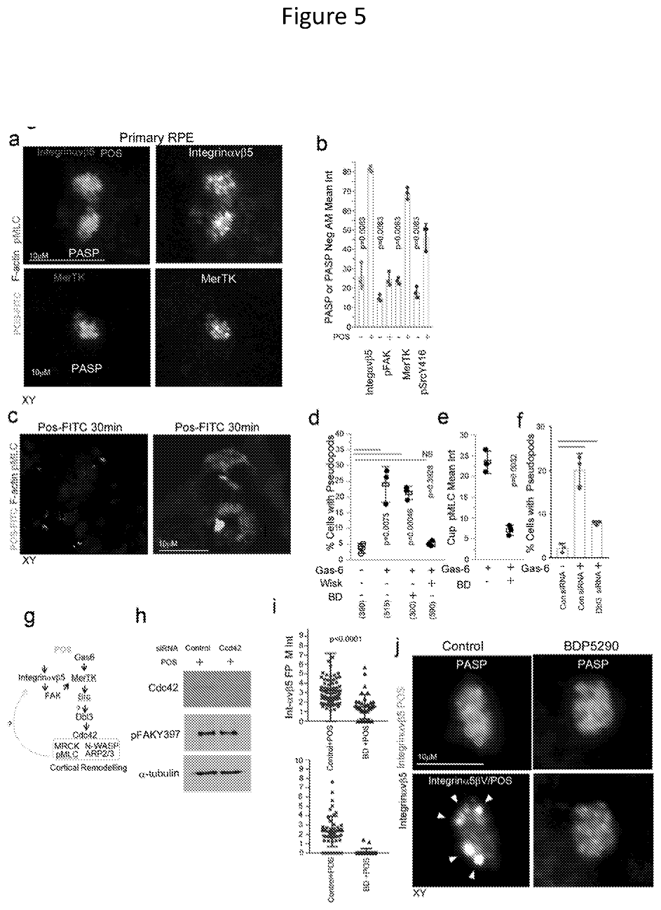

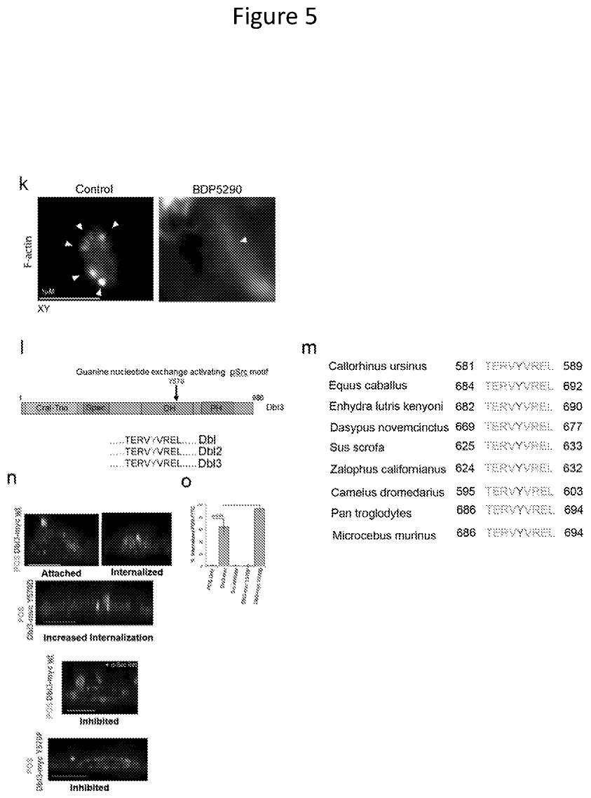

Addition of POS-FITC to porcine RPE for 30 min resulted in PASPs and cup membrane configurations enriched in the integrin, MerTK, and phosphorylated (active) FAK, and c-Src (FIGS. 5a,b). Similar to Cdc42 signalling components receptors signalling components also completely co-localized with POS at the PASP configuration only (FIG. 5a). Thus, POS binding stimulates formation of PASPs containing its receptors with associated effectors as well as the Cdc42 signalling network components.

Since MerTK is required for POS engulfment, the inventors wanted to determine whether the Cdc42 signalling network functions as an intracellular effector signalling plaque of this tyrosine kinase receptor. Primary porcine RPE was cultured in serum-free medium, to eliminate effects of MerTK ligands, for 4 h followed by treatment with either N-WASP or MRCK inhibitor and a 30 min stimulation with POS-FITC in the absence or presence of Gas-6, which links MerTK to POS. POS binding itself did not require Gas-6. However, in cells lacking exogenous Gas-6 POS-dependent pseudopod assembly was inefficient (FIG. 5c, d). When Gas-6 was added together with POS there was a sharp increase in pseudopods along with pMLC staining. Inhibition of N-WASP inhibited pseudopod formation. Unlike POS stimulation with low serum, inhibition of pseudopod formation was more pronounced suggesting that the observed remaining levels in low serum were not due to Gas-6 and that N-WASP signalling functions downstream of Gas-6/MerTK. As expected, inhibition of MRCK did not inhibit pseudopod assembly but pMLC localization at cups (FIG. 5d,e). siRNA-mediated Dbl3 knockdown attenuated pMLC appearance at pseudopods and POS centring (FIG. 5f). Thus, Gas-6 binding to MerTK stimulates a Dbl3-driven Cdc42 signalling network.

Since POS binding to the apical membrane depends on Integrinαvβ5 receptors and consequently the assembly of PASPs the inventors wanted to determine whether signalling to the plaque responds to extracellular stimuli as a mechanical feedback mechanism to guide membrane remodelling around substrate-induced force generation, typically observed in basolateral plaques. To determine whether MerTK-Cdc42 signalling affects biochemical signalling by integrinαVβ5, the inventors knocked down Cdc42 in primary RPE cells and analysed activation of the downstream integrin-effector FAK upon POS addition (FIG. 5h). Levels of FAK phosphorylation were unaffected by inhibiting downstream MerTK signalling. To determine whether Cdc42 signalling affects integrin αvβ5 receptors via mechanical signalling, actomyosin contractility was downregulated by inhibiting MRCK kinase activity and integrin αvβ5 localization was analysed. Inhibition of actomyosin contractility resulted in an inhibition of concentrated spots of integrin αvβ5 localization representing clustering, as well as enrichment of F-actin at corresponding spots at pseudopods (FIG. 5i-k). Therefore, these results indicate that MRCK-driven actomyosin motor activity, which is required for centring of POS via membrane remodelling (FIG. 3a-k) does so by driving integrin αvβ5 localization and clustering around POS as a feedback mechanism between POS attachment and membrane remodelling at attachment sites. Since the activated effector of MerTK, c-Src, colocalizes with the Cdc42 network at PASPs and Dbl3 functions downstream of MerTK, the inventors asked if Dbl3 is activated in response to tyrosine phosphorylation. A bioinformatics analysis using NetPhos3.1 suggested a conserved tyrosine phosphorylation motif TERVYVREL in the Dbl-homology (DH) domain that carries the GEF activity and is completely conserved in diverse mammalian species (FIG. 5l,m). This motif had been shown to be phosphorylated in Dbl1, a splice variant of Dbl3 that lacks the apical targeting information. The TERVYVREL motif of Dbl1 is a Src family kinase target and activates GEF activity. Therefore, a phenylalanine substitution was introduced. ARPE-19 cells expressing Dbl3Y570F-myc were unable to assemble pseudopods that captured and engulfed POS (FIG. 5n,o). Similarly, a Src inhibitor blocked Dbl3-myc induced phagocytosis. Conversely, expression of a tyrosine phosphomimetic Dbl3Y570D mutant increased cup-directed uptake.

Thus, tyrosine-570 is critical for Dbl3-stimulated phagocytosis downstream of MerTK activation.

### Example 7: Dbl3Y570D Restores Phagocytosis in MerTK-Deficient RPE

RNA sequencing of human iPSC-derived RPE cells reveals that short-term inactivation of the Dbl3 pathway leads to increased expression of genes activated in inflammation, retinal stress and RPE fibrosis, and decreased expression of genes associated with RPE differentiation and function (FIG. 17b,c).

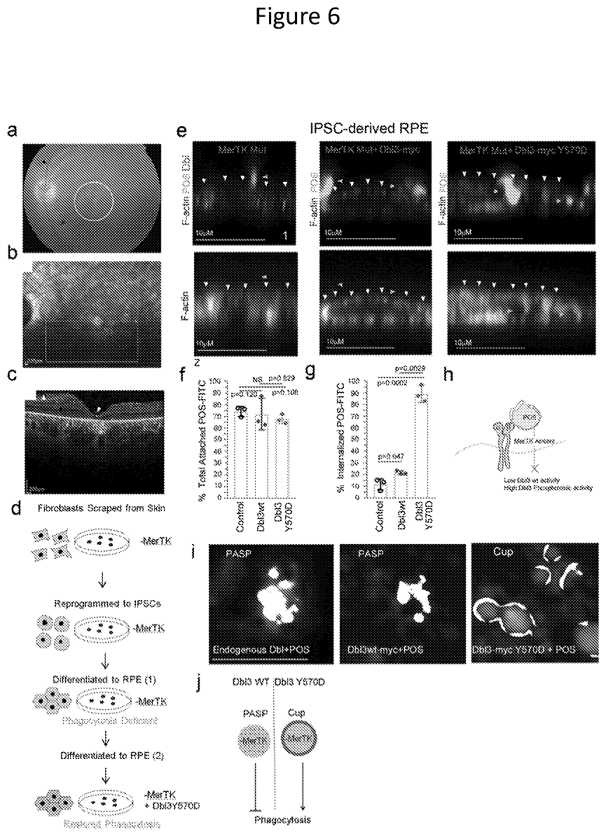

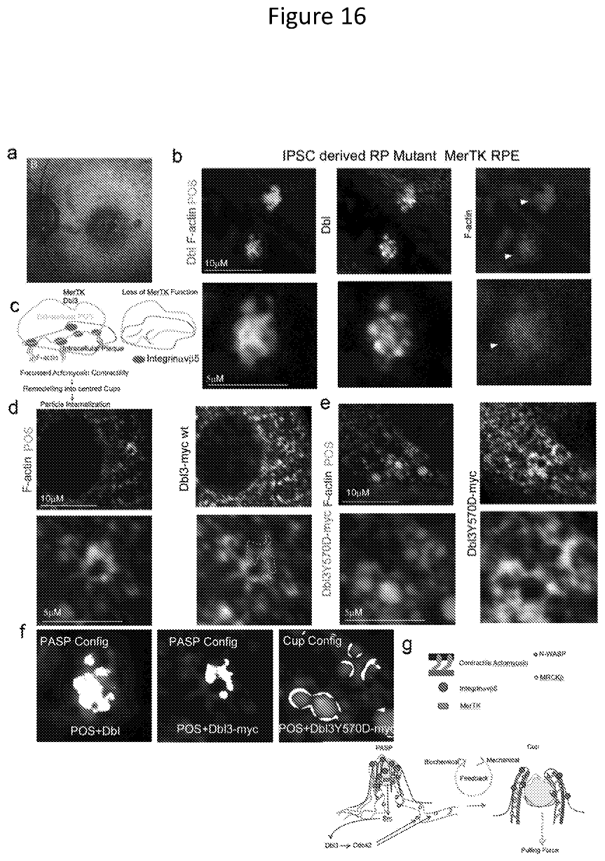

Dbl3 is a key driver of phagocytosis downstream of the cell surface receptors that interact with POS; hence, Dbl3 may be sufficient to stimulate POS internalization in phagocytosis-deficient RPE cells from patients suffering from retinal degeneration. To test this, RPE cells were differentiated from induced pluripotent stem cells (iPSC) derived from a retinitis pigmentosa patient carrying nonsense mutations in the MerTK gene, leading to loss of MerTK protein expression and phagocytosis deficiency (FIG. 6a-c, 6e—left panel; FIG. 16a). These iPSC-derived RPE cells differentiate normally with functional integrin αVβ5 receptors, except for the absence of MerTK protein. As previously reported, these cells displayed poor phagocytosis (˜11%) (FIG. 6e—left panel and 6g). Nevertheless, Dbl co-localized with attached POS in the PASP configuration but did not induce actomyosin contractility-dependent F-actin focal points and did not stimulate the ring configuration (POS-cup contacts) (FIG. 6i; FIG. 16b—left panel; FIG. 16f—left panel), further supporting a primary role for integrin αVβ5 in PASP assembly, and MerTK-Dbl3 signalling in conversion of plaques to cups for internalization.

Expression of Dbl3-myc only weakly increased phagocytosis (˜22%) and, consequently, PASPs remained largely intact. In contrast, expression of Dbl3Y570D increased phagocytosis to 87.0% and promoted efficient transformation of PASPs to phagocytic cups (FIGS. 6i—right panel and 16e and f—right panel).

Thus, Dbl3Y570D expression efficiently rescues phagocytosis in MerTK-deficient RPE cells from an individual suffering from vision loss.

### Example 8: AAV-Mediated Expression of Dbl3 in the RPE Under the Control of a Ubiquitous Promoter Attenuates Loss of Vision in an Animal Model of Inherited Vision Loss

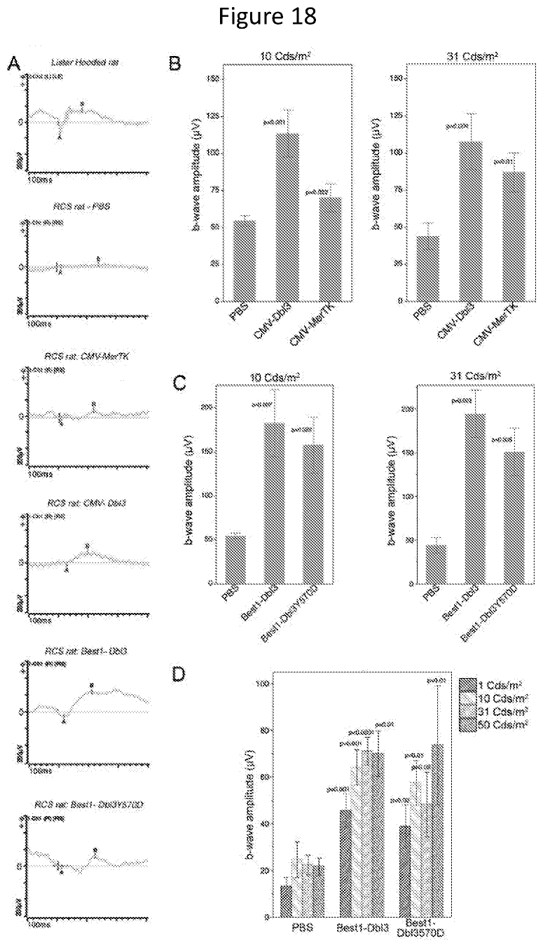

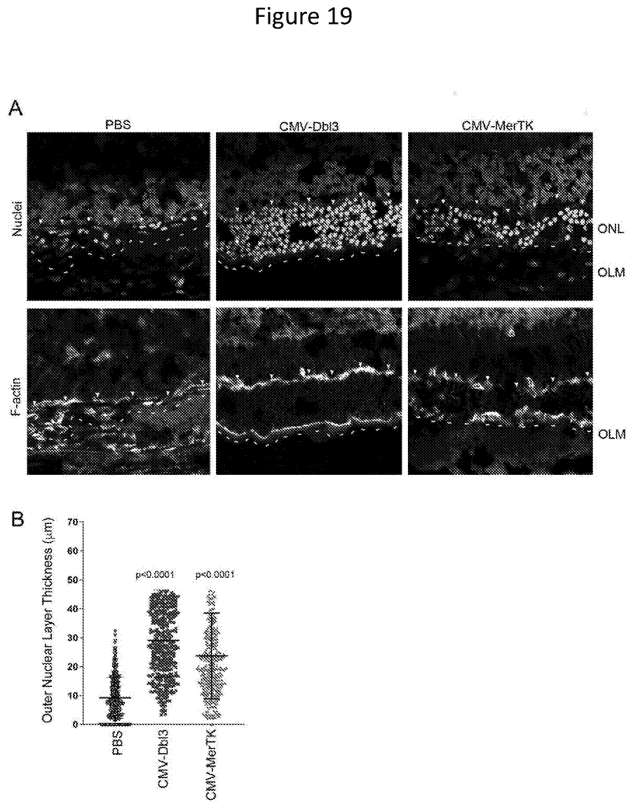

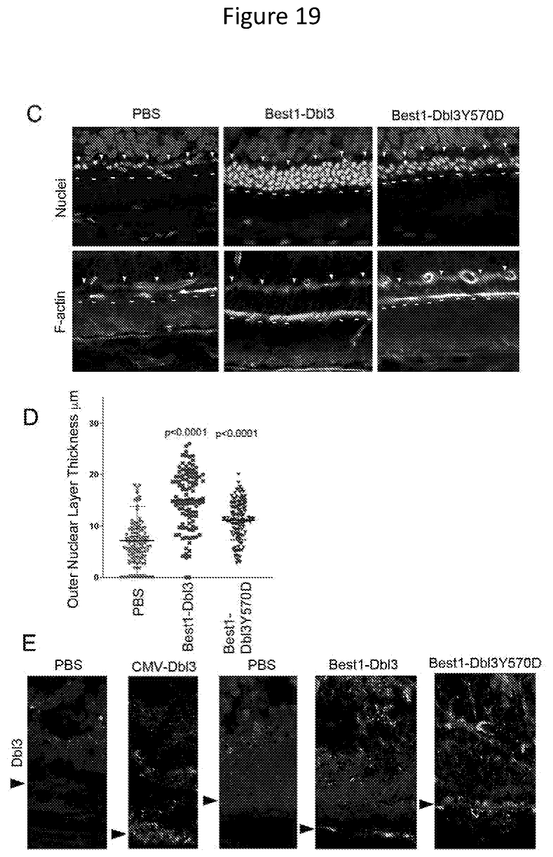

The inventors next asked whether expression of Dbl3 and Dbl3Y570D can rescue loss of vision in an animal model of retinitis pigmentosa, RCS rats. The RCS rat is a well-established model of inherited loss of vision due to a lack of phagocytosis caused by inactivating mutations in the MerTK gene. Retinal degeneration starting within the first weeks after birth shows as a progressive loss of photoreceptors resulting in thinning of the outer nuclear layer (ONL) and defects in the retinal outer limiting membrane (OLM). First, Dbl3 was delivered by subretinal injection of AAV particles, which leads to preferential transduction of the RPE, for expression by a CMV promoter (CMV-Dbl3) and compared to a vector leading to re-expression of MerTK (CMV-MerTK). Both CMV-Dbl3 and CMV-MerTK vectors attenuated loss of retinal function as determined by increased b-wave amplitudes in ERGs comparing to PBS injected control at 6 weeks after injection (FIG. 18A,B). Expression of Dbl3 rescued retinal function as did the expression of MerTK. Analysis of retinal tissue sections using confocal laser scanning microscopy revealed that retinal integrity was greater in both Dbl3 and MerTK transduced RPE when compared to PBS injected retinas, as determined by the thickness of the ONL. The integrity of OLM was also noticeable in CMV-Dbl3 comparing to PBS injected control where it is highly degenerated (FIG. 19A-B, E).

### Example 9: AAV-Mediated Expression of Dbl3 in the RPE Under the Control of an RPE-Specific Promoter Attenuates Loss of Vision in an Animal Model of Inherited Vision Loss

Next, vectors were generated that make use of an RPE-specific promoter. A Best1 promoter fragment (SEQ. ID NO:7) was cloned into AAV vectors along with either Dbl3 (SEQ ID NO: 3) or Dbl3Y570D (SEQ ID NO: 4). Best1-Dbl3 or Best1-Dbl3Y570D AAV vectors were injected subretinally in RCS eyes and retinal function was analysed after 6 weeks or 12 weeks. Both Best-Dbl3 and Best1-Dbl3Y570D vectors rescued retinal function and attenuated loss of vision in RCS rats 6 weeks after AAV injection, as shown by the increased b-wave amplitudes recorded in Best-Dbl3 and Best1-Dbl3Y570D AAV injected RCS eyes comparing to PBS injected controls (FIG. 18A,C). Improved retinal function was also maintained at 12 weeks after injection of either Best-Dbl3 or Best1-Dbl3Y570D AAV vectors (FIG. 18D). The observed values of b-wave amplitudes after injection of Best1-Dbl3 or Best1-Dbl3Y570D particles were as high or higher than what has previously been reported for AAV-mediated expression of MerTK itself using an RPE-specific promoter and similar experimental strategy and methodology (Smith et al., 2003). Thus, expression of Dbl3 and Dbl3Y570D can efficiently rescue retinal function even when the defect is caused by mutations in a different gene. Analysis of retinal tissue using confocal laser scanning microscopy indicated that at 12 weeks after injection, retinal degeneration was strongly attenuated in eyes injected with an AAV vector encoding either Dbl3 or Dbl3Y470D under the control of Best1 promoter, as shown by the greater ONL thickness and presence of OLM in the RPE (FIG. 19C, D, E). Overall, these results demonstrate that expression of Dbl3 or the constitutively active phosphomimetic Dbl3Y470D mutant polypeptide driven by an RPE specific promoter can efficiently prevent or reduce retinal degeneration and loss of retinal function and integrity associated with a defect in phagocytosis.

### REFERENCES

1. Zihni, C. et al. Dbl3 drives Cdc42 signaling at the apical margin to regulate junction position and apical differentiation. J Cell Biol 204, 111-127, doi:10.1083/jcb.201304064 (2014).
2. Zihni, C. et al. An apical MRCK-driven morphogenetic pathway controls epithelial polarity. Nat Cell Biol 19, 1049-1060, doi:10.1038/ncb3592 (2017).
3. Bonilha, V. L. et al. The retinal pigment epithelium apical microvilli and retinal function. Adv Exp Med Biol 572, 519-524, doi:10.1007/0-387-32442-9_72 (2006).
4. Ramsden, C. M. et al. Rescue of the MERTK phagocytic defect in a human iPSC disease model using translational read-through inducing drugs. Sci Rep 7, 51, doi:10.1038/s41598-017-00142-7 (2017).
5. Tsapara, A. et al. The RhoA activator GEF-H1/Lfc is a transforming growth factor-beta target gene and effector that regulates alpha-smooth muscle actin expression and cell migration. Mol Biol Cell 21, 860-870, doi:10.1091/mbc.E09-07-0567 (2010).
6. Bainbridge, J. W. et al. In vivo gene transfer to the mouse eye using an HIV-based lentiviral vector; efficient long-term transduction of corneal endothelium and retinal pigment epithelium. Gene Ther 8, 1665-1668, doi:10.1038/sj.gt.3301574 (2001).
7. Ghazi, N. G., Abboud, E. B., Nowilaty, S. R. et al. Treatment of retinitis pigmentosa due to MERTK mutations by ocular subretinal injection of adeno-associated virus gene vector: results of a phase I trial. Hum Genet 135: 327, doi: 10.1007/s00439-016-1637-y (2016).
8. Maguire et al., Five-Year Outcomes with Anti-Vascular Endothelial Growth Factor Treatment of Neovascular Age-Related Macular Degeneration. Ophthalmology, Volume 123, Issue 8, 1751-1761, doi: 10.1016/j.ophtha.2016.03.045.
9. da Cruz et al., Phase 1 clinical study of an embryonic stem cell-derived retinal pigment epithelium patch in age-related macular degeneration. Nature Biotechnology volume 36, pages 328-337, doi: 10.1038/nbt.4114 (2018).
10. Guidotti G, Brambilla L, Rossi D. Cell-Penetrating Peptides: From Basic Research to Clinics. Trends Pharmacol Sci. 2017 April; 38(4):406-424.
11. B. Davis, E. Normando, L. Guo, P. O'Shea, S. Moss, S. Somavarapu, M. Cordeiro. Topical Delivery of Avastin to the Posterior Segment of the Eye In Vivo Using Annexin AS-associated Liposomes. Small 2014, 10, 1575.
12. Mao Y, Finnemann S C. Regulation of phagocytosis by Rho GTPases. Small GTPases. 2015; 6(2):89-99. doi:10.4161/21541248.2014.989785
13. Gupta M, Qi X, Thakur V, Manor D. Tyrosine phosphorylation of Dbl regulates GTPase signaling. J Biol Chem. 2014; 289(24):17195-17202. doi:10.1074/jbc.M114.573782.
14. Le Meur, G., Stieger, K., Smith, A. et al. Restoration of vision in RPE65-deficient Briard dogs using an AAV serotype 4 vector that specifically targets the retinal pigmented epithelium. Gene Ther 14, 292-303 (2007). https://doi.org/10.1038/sj.gt.3302861.
15. Zihni, C., Mills, C., Matter, K. et al. Tight junctions: from simple barriers to multifunctional molecular gates. Nat Rev Mol Cell Biol 17, 564-580 (2016). https://doi.org/10.1038/nrm.2016.80.
16. Alexander J. Smith, Frank C. Schlichtenbrede, Marion Tschernutter, James W. Bainbridge, Adrian J. Thrasher, and Robin R. Ali AAV-Mediated Gene Transfer Slows Photoreceptor Loss in the RCS Rat Model of Retinitis Pigmentosa MOLECULAR THERAPY Vol. 8, No. 2, August 2003

1. An expression construct comprising a promoter operably linked to a nucleic acid sequence encoding a Dbl3 polypeptide.

2. The expression construct according to claim 1, wherein the Dbl3 polypeptide comprises the sequence of SEQ ID NO: 1, or a sequence with at least 90% sequence identity to the sequence of SEQ ID NO: 1 that maintains the function of the Dbl3 polypeptide.

3. The expression construct according to any one of the preceding claims, wherein said Dbl3 polypeptide is a phosphomimetic; optionally wherein
a) the phosphomimetic mimics phosphorylation of the conserved TERVYVREL activation motif;
b) the tyrosine at amino acid position 570 in SEQ ID NO: 1 is replaced with aspartic acid; and/or
c) the Dbl3 polypeptide comprises the sequence of SEQ ID NO: 2.

4. The expression construct of claim 1, comprising the nucleic acid sequence of SEQ ID NO: 3, or a nucleic acid sequence with at least 90% sequence identity to the sequence of SEQ ID NO: 3 that encodes a protein that maintains the function of the Dbl3 polypeptide; optionally
wherein the nucleic acid sequence comprises the sequence of SEQ ID NO: 4.

5. The expression construct according to any one of claims 1 to 4, wherein the promoter is an RPE-specific or RPE-preferred promoter; optionally
wherein the promoter is selected from a RPE65, BEST1/VDM2, CRALBP1 or TYRP1 promoter, or a fragment thereof that retains the ability to express Dbl3 in the RPE.

6. The expression construct according to claim 5, wherein the RPE-specific promoter comprises:
(a) a sequence of contiguous nucleotides from SEQ ID NO: 7 or SEQ ID NO: 5 that confers RPE-specific expression on an operably linked polynucleotide sequence, or
(b) a sequence having at least 90% sequence identity to said sequence of (a) and that retains RPE-specific promoter activity.

7. The expression construct according to claim 6, wherein the RPE-specific promoter comprises:
(a) at least 400 contiguous nucleotides from SEQ ID NO: 7, or
(b) a sequence having at least 90% sequence identity to said sequence of at least 400 contiguous nucleotides from SEQ ID NO: 7 and retaining RPE-specific promoter activity.

8. The expression construct of claim 6, wherein the RPE-specific promoter comprises:
(a) no more than 800 contiguous nucleotides from SEQ ID NO: 5, or
(b) a sequence having at least 90% sequence identity to said sequence of no more than 800 contiguous nucleotides from SEQ ID NO: 5 and retaining RPE-specific promoter activity.

9. The expression construct of claim 8, wherein the RPE-specific promoter comprises:
(a) the sequence of SEQ ID NO: 6 or the sequence of nucleotides 12-761 of SEQ ID NO: 6, or
(b) a sequence having at least 90% sequence identity to said sequence of SEQ ID NO: 6, or to nucleotides 12-761 of SEQ ID NO: 6, and retaining RPE-specific promoter activity.

10. The expression construct of any one of claims 6 to 9, wherein said promoter of (a) or (b) is at least 500 nucleotides in length.

11. A vector comprising an expression construct according to any one of claims 1 to 10.

12. The vector according to claim 11, wherein the vector is a viral vector; optionally wherein the viral vector is an adeno associated virus (AAV) vector; further optionally wherein the capsid is derived from AAV8.

13. A host cell comprising a vector according to claim 11 or 12.

14. A Dbl3 polypeptide, or a nucleic acid sequence encoding a Dbl3 polypeptide, or an expression construct according to any one of claims 1 to 10, or a vector according to claim 11 or 12, for use in a method for treating retinal dysfunction and/or degeneration.

15. The polypeptide, nucleic acid, expression construct or vector for use according to claim 14, wherein said retinal dysfunction and/or degeneration is characterised by defective phagocytosis of photoreceptor outer segments.

16. The polypeptide, nucleic acid, expression construct or vector for use according to claim 14 or 15, wherein administration of said polypeptide, nucleic acid, expression construct or vector prevents or reduces one or more of the following phenotypes associated with phagocytosis-related retinal degeneration: thinning of the outer nuclear layer (ONL), reduced numbers of internalised phagosomes, accumulation of extracellular shed photoreceptor fragments, impaired formation of pseudopods, irregular apical membrane and reduced cup retraction, altered RPE structural integrity, altered RPE apical membrane signalling, photoreceptor death, Muller cell activation, Bruch's membrane anomalies, deregulated gene expression and loss of vision.

17. The polypeptide, nucleic acid, expression construct or vector for use according to any one of claims 14 to 16, wherein said retinal dysfunction and/or degeneration is an inherited retinal disorder or dystrophy, such as retinitis pigmentosa or macular degeneration; optionally wherein the macular degeneration is age-related macular degeneration (AMD), such as wet or neovascular AMD or geographic atrophy, or inherited dystrophies such as Stargardt and Sorsby diseases.

18. The polypeptide, nucleic acid, expression construct or vector for use according to any one of claims 14 to 17, wherein the Dbl3 polypeptide is defined according to any one of claims 1 to 4.

19. A vector comprising a nucleic acid sequence or an expression construct encoding a gene product that rescues phagocytosis of photoreceptor outer segments, for use in a method of treating retinal dysfunction and/or degeneration.

20. The vector for use according to claim 19, wherein the nucleic acid encodes a Dbl3 polypeptide as defined according to any one of claims 1 to 4, and/or
wherein the vector comprises an expression construct as defined according to any one of claims 1 to 10.

21. The vector for use according to claim 19 or 20, wherein the retinal dysfunction and/or degeneration is inherited retinal disorder or dystrophy, such as retinitis pigmentosa or macular degeneration; optionally
wherein the macular degeneration is age-related macular degeneration (AMD), such as wet or neovascular AMD or geographic atrophy, or inherited dystrophies such as Stargardt and Sorsby diseases.

22. The vector for use according to any of claims 19 to 21, wherein phagocytosis of photoreceptor outer segments is increased following administration of the vector.

23. The vector for use according to any of claims 19 to 22, wherein administration of said vector prevents or reduces one or more of the following phenotypes associated with phagocytosis-related retinal degeneration: thinning of the outer nuclear layer (ONL), reduced numbers of internalised phagosomes, accumulation of extracellular shed photoreceptor fragments, impaired formation of pseudopods, irregular apical membrane and reduced cup retraction, altered RPE structural integrity, altered RPE apical membrane signalling, photoreceptor death, Muller cell activation, Bruch's membrane anomalies, deregulated gene expression and loss of vision.

24. Use of a vector as defined in claim 11 or 12 in the manufacture of a medicament for the treatment of an inherited retinal disorder or dystrophy, such as retinitis pigmentosa or macular degeneration; optionally
wherein the macular degeneration is age-related macular degeneration (AMD), such as wet or neovascular AMD or geographic atrophy, or inherited dystrophies such as Stargardt and Sorsby diseases.

25. A method of improving vision in a patient with retinal dysfunction and/or degeneration by introducing into the RPE a vector comprising a nucleic acid encoding a Dbl3 polypeptide; optionally
wherein the vector is as defined in claim 11 or 12; and/or
wherein vision is improved as defined in any one of claims 21 to 23.
